(19) United States
(12) Patent Application Publication
(10) Pub. No.: US 2024/0075295 A1
(43) Pub. Date: Mar. 7, 2024

(54) METHODS OF MANEUVERING STIMULATION ELEMENTS WITHIN VESSELS VIA A GUIDE TOOL

(71) Applicant: INSPIRE MEDICAL SYSTEMS, INC., Golden Valley, MN (US)

(72) Inventor: Wondimeneh Tesfayesus, New Brighton, MN (US)

(73) Assignee: INSPIRE MEDICAL SYSTEMS, INC., Golden Valley, MN (US)

(21) Appl. No.: 18/239,907

(22) Filed: Aug. 30, 2023

Related U.S. Application Data
(60) Provisional application No. 63/403,038, filed on Sep. 1, 2022.

(51) Int. Cl. A61N 1/36; A61B 17/34

(52) U.S. Cl. CPC A61N 1/3611; A61B 17/3468

(57) ABSTRACT

A system and/or method involving establishing, via a guide tool, a stimulation element in a first position within a first vessel of a vasculature which is in communication with a second vessel of the vasculature, identifying, via angularly positioning of an elongate arm relative to a distal elongate portion of the guide tool, an entry region into the second vessel from the first vessel, and maneuvering the stimulation element through the entry region and to a second position within the second vessel via the guide tool.

### CROSS-REFERENCE TO RELATED APPLICATIONS

This application claims the benefit of the filing date of U.S. Provisional Application Ser. No. 63/403,038, filed Sep. 1, 2022 and entitled “Methods of Maneuvering Stimulation Elements within Vessels via a Guide Tool,” the entire teachings of which are incorporated herein by reference.

### BACKGROUND

Many patients benefit from therapy provided by an implantable medical device (IMD). For example, a portion of the population suffers from various forms of sleep disordered breathing (SDB), such as sleep apnea. Sleep apnea generally refers to the cessation of breathing during sleep. One type of SDB, referred to as obstructive sleep apnea (OSA), is characterized by repetitive pauses in breathing during sleep due to the obstruction and/or collapse of the upper airway, and is usually accompanied by a reduction in blood oxygenation saturation.

One treatment for SDB includes the delivery of electrical stimulation to the hypoglossal nerve, located in the neck region under the chin. Such stimulation therapy activates the upper airway muscles to maintain upper airway patency. In treatment of SDB, increased respiratory effort resulting from the difficulty in breathing through an obstructed airway is avoided by synchronized stimulation of an upper airway muscle or muscle group that holds the airway open during the inspiratory phase of breathing. For example, the genioglossus muscle is stimulated during treatment of SDB by a cuff electrode placed around the hypoglossal nerve.

### BRIEF DESCRIPTION OF THE DRAWINGS

FIG. 1 is a flow diagram schematically representing an example method of maneuvering a stimulation element through a vasculature via a guide tool.

FIG. 2 illustrates an example guide tool.

FIGS. 3A-3E are diagrams schematically representing an example operation of a guide tool.

FIGS. 4A-4E are diagrams schematically representing another example operation of a guide tool.

FIG. 5A illustrates an example IMD.

FIG. 5B is a diagram schematically representing patient anatomy and an example device and/or example method for stimulating an infrahyoid muscle (IHM)-innervating nerve, hypoglossal nerve, and/or other target tissue.

FIGS. 6A-9B are diagrams, which may comprise part of a flow diagram in an example method.

FIGS. 10A-10G are diagrams schematically representing an example of maneuvering a guide tool through a vasculature.

FIGS. 11A-11B are schematic illustrations of vasculature placement of a stimulation element of an IMD.

FIGS. 12A-12B are schematic illustrations of delivering stimulation via multiple vessels of a vasculature.

FIGS. 13A-13B are illustrations of example devices comprising IMDs.

FIGS. 14A-18D illustrate example variations of guide tools and/or IMDs.

FIG. 19A is a block diagram schematically representing an example control portion.

FIG. 19B is a diagram schematically illustrating at least some example arrangements of a control portion.

FIG. 20 is a block diagram schematically representing a user interface.

FIG. 21 is a block diagram schematically representing example implementations by which an IMD may communicate wirelessly with external circuitry outside the patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

Examples of the present disclosure are directed to providing stimulation to tissue for treating SDB. For example, an implantable medical device (IMD) may be used to provide stimulation to a hypoglossal nerve, or other target tissue, through a intravascular lead system. The lead system may include at least one stimulation element used to provide stimulation.

In some examples, the stimulation may be provided synchronously with respiration detected by a sensing lead system. In some examples, a single lead includes both a sensing lead and the stimulation lead, and in other examples, the sensing lead forms a lead separate from a stimulation lead. In some examples, the sensing lead comprises a sensing lead external to the vasculature (such as being mounted externally on a patient or subcutaneously implanted) while the stimulation lead comprises an intravascular lead. In some examples, sensing can be provided via an on-board sensor incorporated into an implantable pulse generator, such as an accelerometer or electrodes for sensing. In some such examples, a separate sensing lead is omitted.

To provide the stimulation, the stimulation element may be intravascularly placed within a vessel of the vasculature in a position that is adjacent to the target tissue. As such, the stimulation element is in close proximity to the target tissue and stimulation may be delivered transvascularly to the target tissue via the stimulation element. Maneuvering the stimulation element or other tools within the vasculature and adjacent to the target tissue is challenging due to physiological features of the vasculature and physiological differences between patients. For example, identifying the entry region into the vessel from another vessel may be difficult due to the angle that the two vessels intersect at, the diameter of the vessels (e.g., around 2 mm), and/or the spacing therebetween. A physician (e.g., surgeon) may be unable to properly place the stimulation element and/or make take hundreds of attempts to place the stimulation element, which increases fatigue and risk for error. Some delivery techniques involve the use of a contrast agent which is observed using an imaging system to guide placement of the stimulation element. In some examples, the vessel adjacent to the target tissue includes a lingual-valvous vessel. As used herein, a lingual-valvous vessel includes and/or refers to a lingual vessel that includes valves. The valves allow for fluid flow in a first direction and prevent fluid flow in a second direction, and may not allow for the contrast agent to travel through the vessel. Maneuvering through a lingual-valvous vessel may thus be performed blindly or by feel, which increases the difficulty.

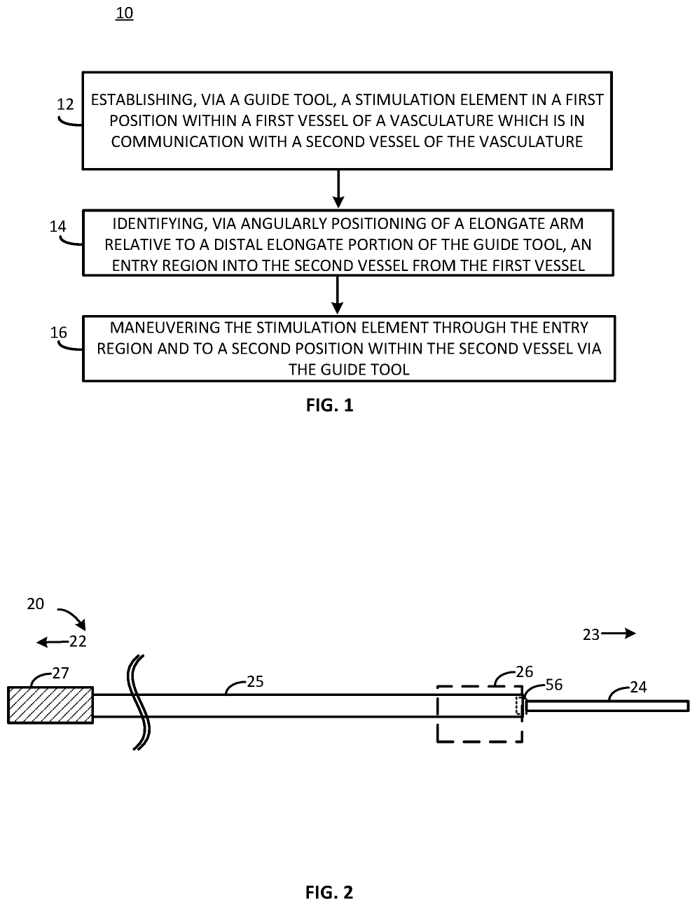

Examples of the present disclosure are directed to a guide tool and methods of using the guide tool to maneuver a stimulation element within a vasculature. The guide tool includes an elongate arm which may accentuate palpation to identify surfaces within the vasculature. Such surfaces may include topographic structures, such as entry regions between respective vessels of the vasculature and valves within respective vessels which are lingual-valvous vessels. Anatomical topographic structures include and/or refer to internal lining or walls of the vessel, entry regions, and valves. An entry region includes and/or refers to a connection or entrance between vessels, which provides fluidic communication between the vessels. These examples, and additional examples, are described in association with at least FIGS. 1-21.

As used herein, vessels include and/or refer to channels or conduits through which blood is distributed to body tissue. Vessels are sometimes interchangeably referred to herein as veins. Vasculature includes and/or refers to the vascular system of a patient, including the arrangement of the vessels forming the vascular system and connecting the heart with other organs and tissues.

FIG. 1 is a flow diagram schematically representing an example method 10 of maneuvering a stimulation element through a vasculature via a guide tool. The vasculature may comprise a non-cardiac vasculature including lingual vessels. At least one of the lingual vessels may have valves and is located proximate to a target tissue for providing stimulation to treat a patient, such as treating SDB. As further described herein, in some examples, the target tissue may comprise an upper airway patency-related tissue such as, but not limited to, an infrahyoid muscle (IHM)-innervating nerve, an infrahyoid muscle (IHM) (e.g., infrahyoid strap muscle), a hypoglossal nerve, a genioglossus muscle, an internal superior laryngeal (iSL) nerve, a glossopharyngeal nerve, pharyngeal muscles, and/or other nerves/muscles of the upper airway. In some examples, the target tissue for treating sleep disordered breathing may comprise a phrenic nerve and/or a diaphragm muscle.

At 12, the method 10 includes establishing, via a guide tool, a stimulation element in a first position within a first vessel of a vasculature. The first vessel may be in communication with a second vessel of the vasculature. Establishing the stimulation element in the first position may include percutaneously inserting the guide tool through the skin surface (and subcutaneous tissues) to establish the stimulation element and/or guide tool within the vasculature and then advancing the guide tool within and through the vasculature (e.g., intravascularly advancing) to the first position within the first vessel. The stimulation element includes and/or refers to at least one stimulation electrode that provides a stimulation signal. Example stimulation elements include an electrode(s), electrode arrays, ring electrode(s), cuff electrodes, among others.

At 14, the method 10 includes identifying, via angularly positioning of an elongate arm relative to a distal elongate portion of the guide tool, an entry region into the second vessel from the first vessel. As further shown by FIG. 2 and referring to FIG. 2, the distal elongate portion 26 includes a linear segment of the elongate portion 25 of the body of the guide tool 20 that is proximate and/or coupled to the elongate arm 24, and that remains aligned with the elongate arm 24 when the elongate arm 24 is at least relatively straight or non-angled (e.g., is in a first state or locked in angular position) with respect to a longitudinal axis of the distal elongate portion 26. The entry region may be identified by tilting and/or rotating the elongate arm. For example and referring back to FIG. 1, the method 10 may comprise angularly positioning the elongate arm at or to least one of: i) a tilt angle relative to the distal elongate portion of the guide tool, and ii) a rotational angle relative to a longitudinal axis of the distal elongate portion, as further described herein.

At 16, the method 10 includes maneuvering the stimulation element through the entry region and to a second position within the second vessel via the guide tool, where the second position is adjacent to a target tissue. The target tissue may include a nerve and/or muscle, such as the hypoglossal nerve. Other non-limiting example target tissue includes the phrenic nerve, an IHM-innervating nerve, among other nerves and muscles, as more fully described later.

The entry region may comprise a junction (e.g., an ostium) of or between the first vessel and the second vessel. In some examples, maneuvering the stimulation element comprises inserting the guide tool along a pathway through the vasculature from the first vessel to the second vessel by inserting and advancing the stimulation element through the entry region. In some examples, the entry region is identified and the stimulation element is maneuvered through the entry region and to a second position within the second vessel without anchoring the distal portion (e.g., the elongate arm) of the guide tool at the entry region to the second vessel.

In some examples, the second vessel extends at a first angle relative to the first vessel at the entry region. The first angle may include an acute angle, a right or perpendicular angle, or an obtuse angle. Different patients may exhibit different first angles between the first and second vessels. In some examples, the first angle may be less than about 180 degrees. In some example, the first angle may be between about 30 degrees and about 150 degrees, about 45 degrees and about 135 degrees, about 60 degrees and about 120 degrees, about 70 degrees and about 110 degrees, or about 80 degrees and about 100 degrees, among other variations. In some examples, portions of the first vessel and/or the second vessel which are not at the entry region (e.g., junction) may define a second angle relative to one another that is different from the first angle at the entry region.

The entry region and other surfaces may be identified via accentuated palpation (e.g., feeling) enabled by the elongate arm and/or via sensing provided by the elongate arm of the guide tool. The surfaces may include entry regions, valves, and/or other topographic structures. In some examples, the guide tool may accentuate palpation, as compared to a guide wire or other delivery tool, by accentuating changes in pressure or tension on the guide tool due to the entry region, valve, and/or other topographic structure. For example, when the elongate arm advances over the entry region, the pressure or tension on the guide tool may be reduced. When the elongate arm contacts a valve, the pressure or tension on the guide tool may increase. In many instances, identification of an entry region or valve may be made by a sudden change in pressure, tension, and/or other feedback at the elongate arm of the guide tool. The feedback may be mechanical or sensed via a sensor, such as an impedance, light, pressure, or and/or sound (e.g., sonographic, ultrasonic, among others) sensor(s). Once the entry region is identified, a threshold amount of force sufficient to cross or push through valve(s) may be applied to the lead, such that the elongate arm may advance through the valve(s) in the vessel. In some examples, the palpable contact of the elongate arm with the surfaces is transmitted from the elongate arm to a handle (e.g., 27 of FIG. 2) of the guide tool that may be gripped by a clinician.

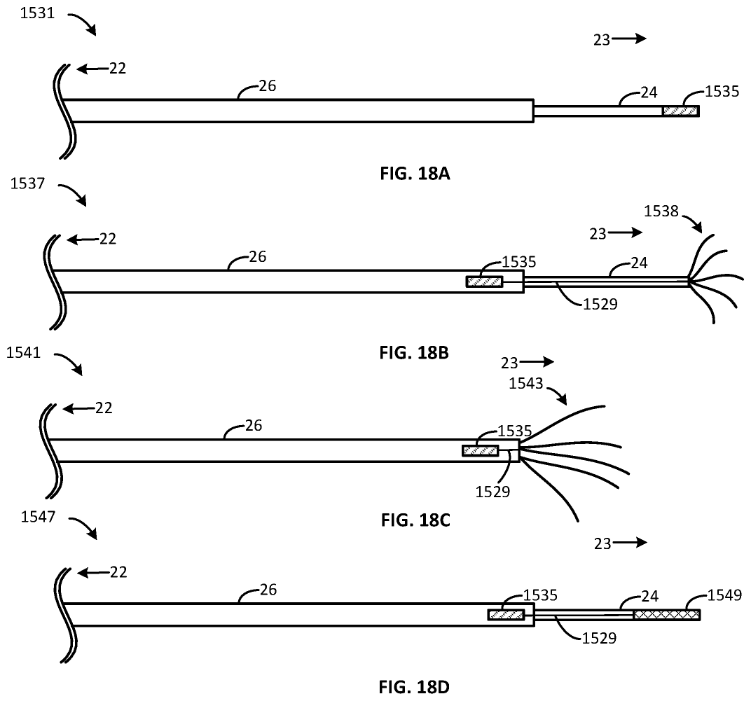

As an example, the elongate arm may enable accentuated palpation or sensing of topographic structures based on changes in pressure which may be caused by or in response to a transition from a vessel wall of the first vessel to an opening or hole, which is the entry region to the second vessel from the second vessel. The transition (e.g., change in pressure) may include and/or result in detecting a rim of the hole (e.g., the entry region) where the tip of the elongate arm goes from contacting the vessel wall, with a felt or sensed pressure, to extending into the hole or a void, resulting in a sudden change (e.g., decrease) in force and/or pressure perceived via the handle (e.g., 27 of FIG. 2) or sensed via a sensor (e.g., 1535 of FIG. 18A). Similarly, the transition (e.g., change in pressure) may include and/or result in detecting a valve where the tip of elongate arm goes from contacting the vessel wall of the second vessel or not contacting a topographic structure to contacting the valve, resulting in a sudden change (e.g., increase) in force and/or pressure perceived via the handle (e.g., 27 of FIG. 2) or sensed via a sensor (e.g., 1535 of FIG. 18A). The force felt or sensed in response to contact of the elongate arm with a valve may be greater than a threshold, and may involve applying pressure or push with a threshold amplitude by the guide tool to proceed through the valve. In some examples, the clinician may count the number of valves as the guide tool is moved through the second vessel, which may provide feedback on how far the guide tool has proceeded into and through the second vessel.

In some examples, maneuvering the stimulation element through the entry region and to the second position within the second vessel comprises advancing the distal elongation portion of the guide tool proximate to a target location within the second vessel adjacent to the target tissue via a pathway through the second vessel, and positioning the stimulation element proximate to the target location via the guide tool. The target location may be sufficiently close to the target tissue to deliver stimulation signals to the target tissue and stimulate the target tissue. In some instances, with the stimulation element at the target location, the stimulation element may be said to be in stimulating relation to the target tissue. Maneuvering the stimulation element may comprise maneuvering through the entry region and to the second position within the second vessel by advancing the elongate arm through the entry region and through at least one valve within the second vessel.

In some examples, maneuvering the stimulation element through the entry region and to the second position in the second vessel comprises at least one of: i) advancing the guide tool into and through a subclavian vein, into and through a jugular vein, into and through a vein trunk, and into the vena comitante hypoglossi (VCH), and ii) advancing the guide tool into and through a common facio-lingual branch, into and through a lingual vein, and into a ventral lingual vein. For example, the target tissue may comprise the hypoglossal nerve and the second vessel comprises the VCH, sometimes referred to as the “ranine vein”. For example, maneuvering the stimulation element through the entry region and to the second position in the second vessel may comprise advancing the distal elongation portion of the guide tool into the entry region and through at least one valve of the VCH.

Examples may be directed to other types of vessels, such as but not limited to other vessels associated with the hypoglossal nerve, an IHM-innervating nerve, iSL nerve, glossopharyngeal nerve, phrenic nerve, and/or other nerves, as more fully described later.

In some examples, the guide tool is maneuvered through the second vessel in a direction that is opposite to blood flow. As described above, the valves in the second vessel may be positioned to allow fluid flow in one direction and to prevent fluid flow in the opposite direction. The guide tool may provide sufficient pressure to push through the valves, which are positioned to prevent fluid from traveling in the direction that the guide tool is being maneuvered.

Without use of an elongate head, it may take dozens or more tries to find and enter the entry region to the second vessel from the first vessel. By having an elongate arm that may be moved to different angular positons, e.g., is not fixed or not fixed at particular times, the entry region may be identified more quickly and/or with only use of a single entry region as compared to a fixed tip or tip at a fixed angle, which may reduce recovery time, reduce operation time, and reduce risk of complications to the patient as compared to identifying the entry region without use of the elongate head.

In some examples, the stimulation element may be maneuvered to the second position within the second vessel and the method 10 may further comprise transvascularly (e.g., through a wall of the vessel) delivering stimulation to the target tissue via the stimulation element. For example, the target tissue may comprise an upper airway-related tissue and the method 10 may comprise transvascularly delivering stimulation to the target tissue via the stimulation element to treat SDB. In some examples, the method 10 comprises positioning the stimulation element within the second vessel at the second position in close proximity to a hypoglossal nerve and within a neck region of the patient, which may be used for treating SDB.

In some examples, the method 10 further comprises detecting a SDB event for the patient, and transvascularly delivering the stimulation via the stimulation element in response to the detection of the SDB event, wherein transvascularly delivering the stimulation element to the target tissue causes an increase or maintaining of patency of an upper airway of the patient.

As further described herein, the method 10 may include a number of additional steps and/or variations, such as different types and variations of guide tools and methods of maneuvering the stimulation element to the position.

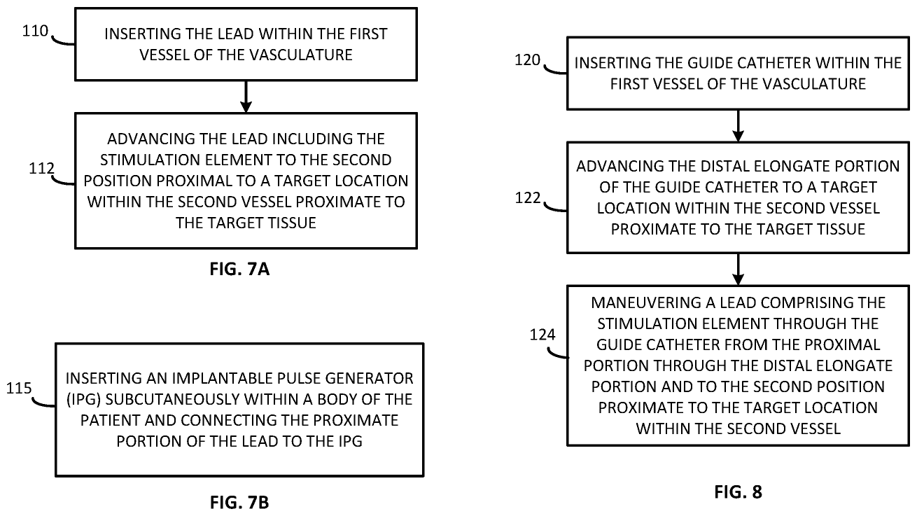

In some examples, the method 10 further comprises implanting an implantable pulse generator (IPG) subcutaneously at a pectoral, non-vasculature location within a body of the patient, and connecting a lead to the IPG, wherein the lead includes the stimulation element that is in electrical communication with the IPG. For example, the guide tool may comprise a lead which is connectable to an IPG to form an IMD, as further described by FIG. 7A.

In other examples, the guide tool comprises a guide catheter and a lead is maneuvered through the guide catheter, as further described by FIG. 8.

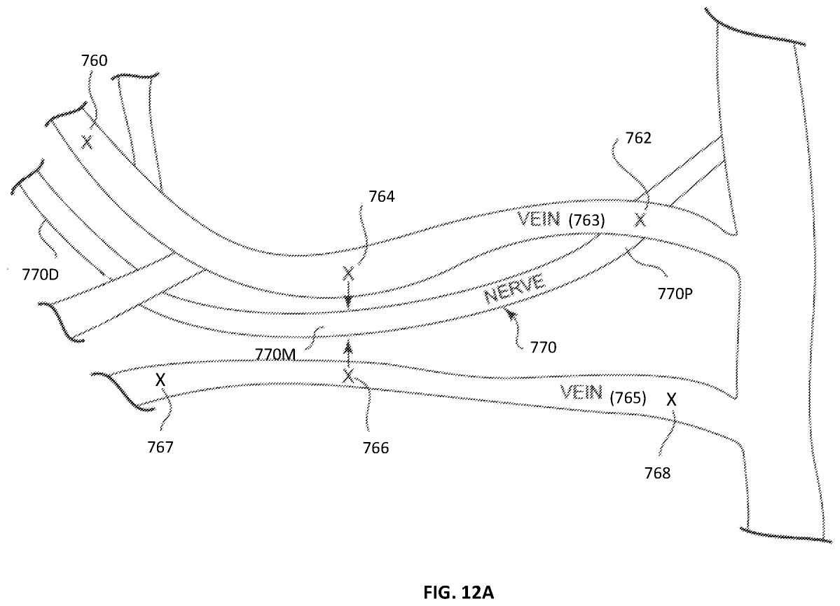

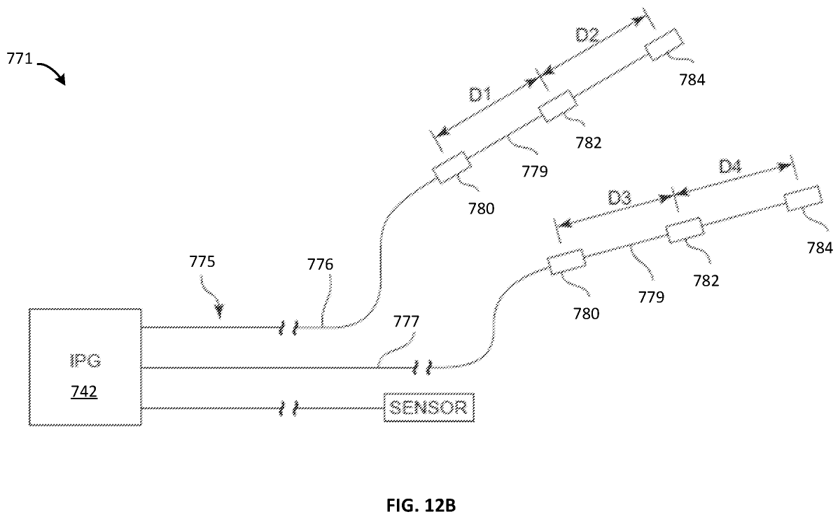

In some examples, the stimulation element comprises a first stimulation element, and the guide tool comprises a first guide tool. The method 10 may further comprise establishing, via a second guide tool, a second stimulation element in a third position within the first vessel or a fourth vessel of the vasculature which is in communication with a third vessel of the vasculature, identifying, via angular positioning of an elongate arm of a distal elongate portion of the second guide tool, an entry region into the third vessel from the first vessel or the fourth vessel, and maneuvering the stimulation element through the entry region and to a fourth position within the third vessel via the second guide tool, the fourth position being adjacent or proximate to the target tissue, as further illustrated in FIGS. 12A-12B.

FIG. 2 illustrates an example guide tool 20. The guide tool 20 may be used in the method 10 of FIG. 1, in various examples. As shown, the guide tool 20 includes a proximal portion (or end) 22, a distal portion 23, and an elongate portion 25 between the proximal portion 22 and the distal portion 23, which form a body of the guide tool 20. The distal portion 23 comprises an elongate arm 24. The elongate portion 25 includes a distal elongate portion 26 that is coupled to the elongate arm 24 on the distal portion 23 of the guide tool 20. The proximal portion 22 may include a handle 27 for a clinician to hold.

The guide tool 20, including the body, may be solid or hollow, and the elongate portion 25 may extend a length sufficient to extend through a head-and-neck region of the patient and diameter that is smaller than a diameter of a vessel, such as less than 2 mm or less than 1 mm. In some examples, the guide tool 20 may be implemented as or include a guide wire with sensing capabilities. The guide tool 20 may be shaped such that the elongate portion 25 and the elongate arm 24 are pushable and torquable, and the elongate arm 24 is steerable, each of which enables maneuvering the guide tool 20 within and/or through the vasculature. The body, including the portions 22, 23, 25, may be generally flexible and resilient member(s), and the elongate arm 24 may have the same or different stiffness than the elongate portion 25 and/or the distal elongate portion 26. In some examples, the elongate arm 24 may have varying stiffness, such as being stiffer at a proximal portion of the elongate arm 24 than a distal portion, and in other examples, may have uniform thickness throughout. As further described herein, the elongate arm 24 may be shorter than the elongate portion 25 and/or may be longer or shorter than the distal elongate portion 26. The elongate arm 24 may have a variety of different shapes and/or sizes, such as an elongate cylindrical or elliptical shape, among other shapes.

The guide tool 20 may be in different states associated with the angular position of the elongate arm 24 with respect to the distal elongate portion 26 depending on a position of the elongate arm 24. FIG. 2 illustrates a first state of the guide tool 20 in which the elongate arm 24 is aligned with a longitudinal axis of the distal elongate portion 26. The guide tool 20 may be in a second state in which the elongate arm 24 is unaligned with the longitudinal axis of the distal elongate portion 26 or is otherwise tilted with respect to the longitudinal axis, sometimes referred to as a tilt angle. As further described herein, the elongate arm 24 may be positioned or moved at or to at least one of a tilt angle relative to the distal elongate portion 26 of the guide tool 20, and a rotational angle relative to or about a longitudinal axis of the distal elongate portion 26.

As further described below, the elongate arm 24 may be movable among or between different angular positions according to a degree of freedom and/or multiple degrees of freedom, which may be expressed as being different orientations. In some examples, the elongate arm 24 may be movable in a 180 degree range with respect to a longitudinal axis of the distal elongate portion 26, which may sometimes be referred to as tilting the elongate arm 24 and in a tilt range or a tilt angle (e.g., 180 degrees or other ranges). Examples include other tilt degree ranges, such as 360 degrees, 300 degrees, 280 degrees, 170 degrees, 150 degrees, among others. In some examples, the elongate arm 24 may be movable in a 360 degree range about the longitudinal axis of the distal elongate portion 26, which may sometimes be referred to as rotating the elongate arm 24 and in a rotation range or angle (e.g., 360 degrees or other ranges). In some such examples, the elongate arm 24 may be positioned in any degree within the range(s). In some examples, the elongate arm 24 may be moved within a tilt range and within a rotation range.

In some examples, the elongate portion 25 may be rotatable, in addition to tilting and/or rotating the elongate arm 24. For example, the clinician may rotate the elongate portion 25, and thereby rotate the elongate arm 24, via the handle 27, which may be used to maneuver the guide tool 20 through the vasculature. In some such examples, the elongate arm 24 may be at a tilt angle and/or moved to multiple tilt angles while the guide tool 20 is being rotated about a longitudinal axis of the elongate portion 25 by the clinician. In some examples, once the distal elongation portion 26 is proximate to a target region, such as to or near the entry region, the elongate portion 25 may not be rotated and/or may remain generally stable and the elongate arm 24 is moved to different angular positions relative to the temporarily stable elongate portion 25.

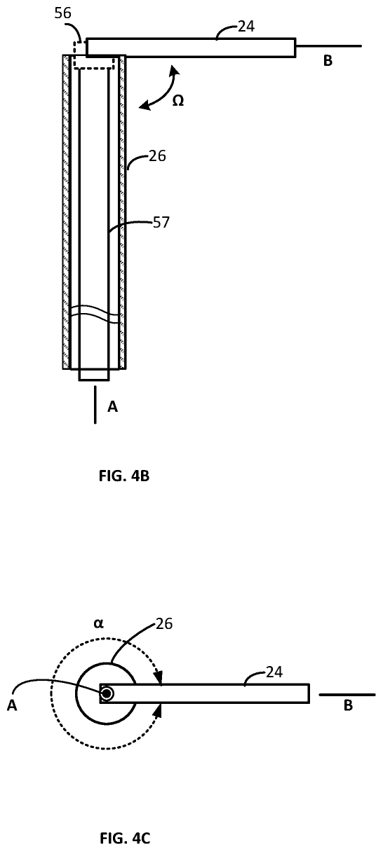

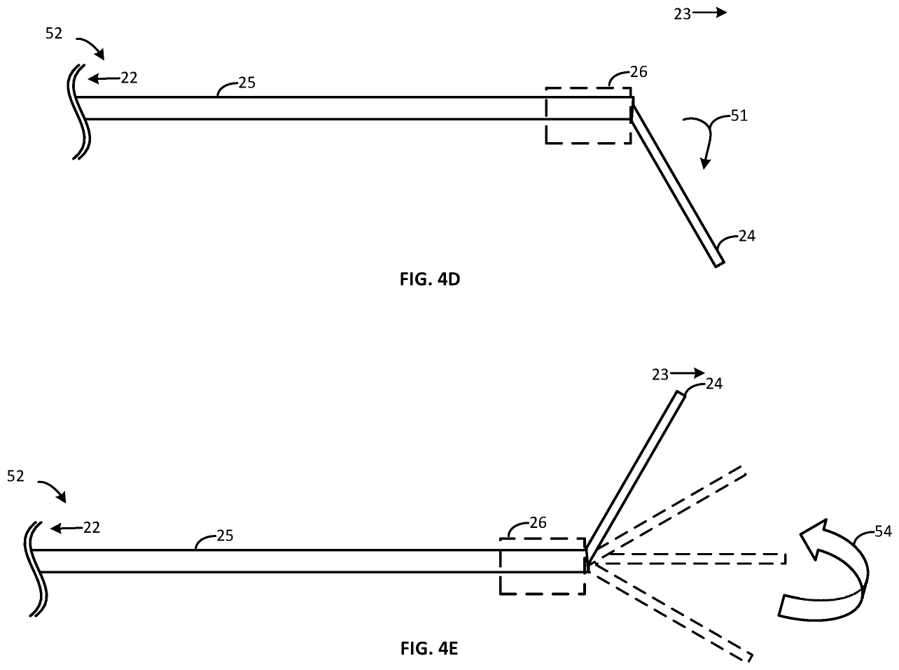

In some examples, once the distal elongation portion 26 is proximate to a target region, such as extending through the entry region and into the second vessel, the elongate arm 24 may momentarily or temporarily remain in a stable position, such as locking the elongate arm 24 in a position in which the elongate arm 24 is generally straight or non-angular relative to the distal elongate portion 26 (sometimes herein referred to as a “first state”), which may enhance sensing palpable pressure in response to contacting valve(s) and/or enable greater pressure for pushing through the valve(s). In some examples, after pushing the elongate arm 24 through a valve (and in response to decrease pressure sensed or felt), the elongate arm 24 may be moved to an angular position relative to the distal elongate portion 26 (sometimes referred to as a “second state”). In some examples, as further illustrated by FIG. 4B, a wire 57 or other mechanism may extend longitudinally through the elongate portion 25 to enable the elongate portion 25 to remaining stable (e.g., not rotate) at times.

Referring back to FIG. 2, in some examples, the proximal portion 22 of the guide tool 20, such as the handle 27, may include a mechanism to permit the clinician to control the angular position of the elongate arm 24. The mechanism may be an electrical button, mechanical wire, or other mechanisms, which may cause the change in angular positioning, such as by activating an actuator that moves the junction (e.g., hinge) coupled to the elongate arm 24. In some such examples, the guide tool 20 further includes a junction 56, such as a joint or other miniaturized rotational mechanism contained within the junction 56 which enables the angular position of the elongate arm 24. For example, the miniaturized rotational mechanism may be actuated in response to electrical signals sent through electrical wires responsive to selection of an electrical button on the handle 27 by the clinician. As further described herein, the junction 56 may include a hinge joint, a pivot joint, and/or a ball joint that forms part of the distal elongate portion 26 and that couples to the elongate arm 24. For example, the elongate portion 25 may include electrically conductive wires which extend through and within the body of the guide tool 20 and connect the mechanism in the handle 27 to the junction 56.

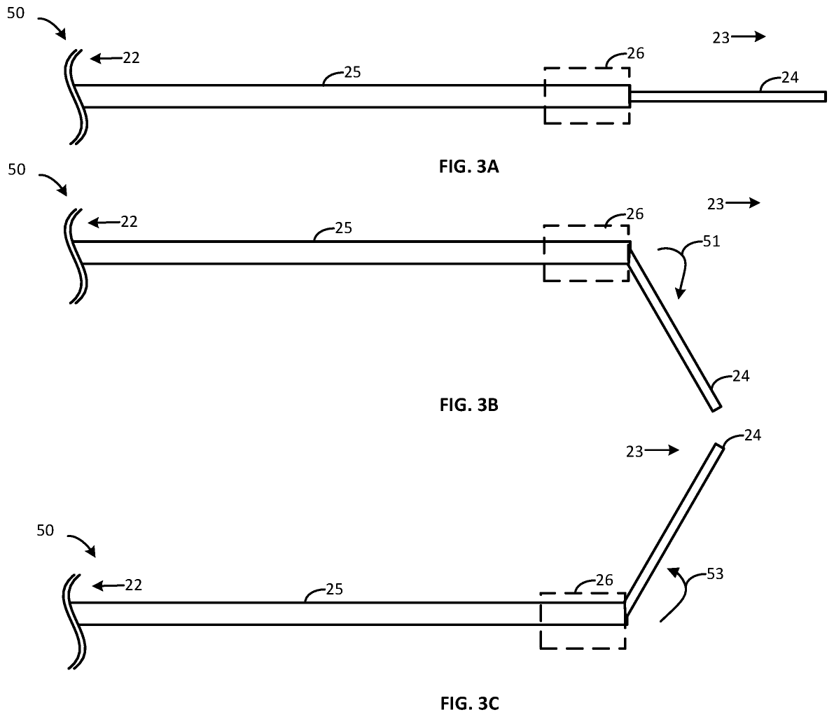

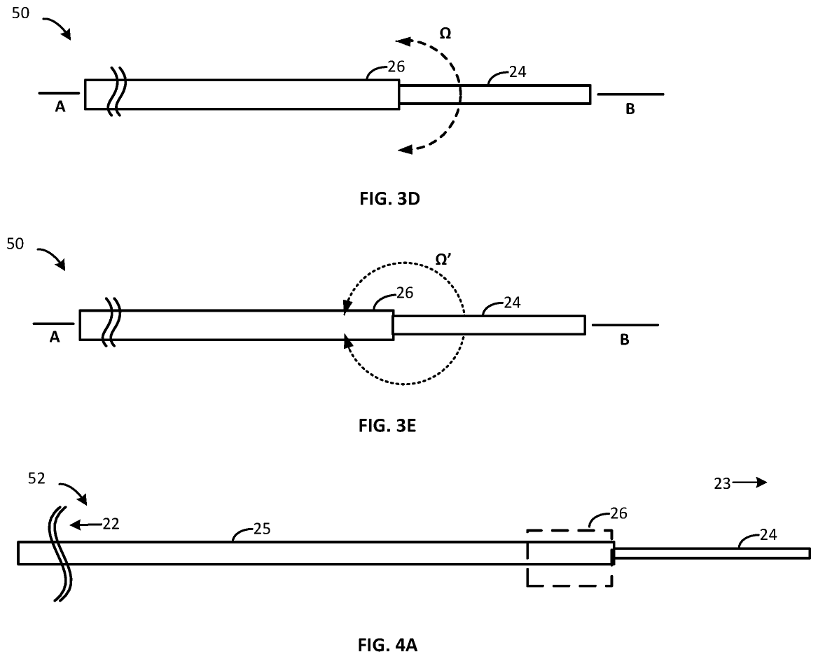

FIGS. 3A-3E are diagrams schematically representing an example operation of a guide tool. The guide tool 50 of FIGS. 3A-3E may include at least some of substantially the same features and attributes as the guide tool 20 of FIG. 2, the common features and attributes not being re-described below.

FIG. 3A illustrates an example of a first state of the guide tool 50 in which the elongate arm 24 is aligned with the longitudinal axis of the distal elongate portion 26. FIGS. 3B-3C illustrate a second state of the guide tool 50 in which the elongate arm 24 is unaligned with the longitudinal axis (e.g., A as shown by FIGS. 3D-3E) of distal elongate portion 26. Being aligned with the longitudinal axis of the distal elongate portion 26, as used herein, includes and/or refers to the longitudinal axis of the elongate arm 24 (e.g., B as shown by FIGS. 3D-3E) being aligned with the longitudinal axis of the distal elongate portion 26 (e.g., A as shown by FIGS. 3D-3E), such that the elongate arm 24 is generally straight with respect to the distal elongate portion 26. Unaligned with the longitudinal axis of the distal elongate portion 26 includes and/or refers to the longitudinal axis of the elongate arm 24 being unaligned with the longitudinal axis of the distal elongate portion 26, such that the elongate arm 24 is at an angle (e.g., a tilt angle) with respect to the distal elongate portion 26. For example, in the second state, the respective longitudinal axes of the distal elongate portion 26 and the elongate arm 24 are in divergent orientations and/or may not align with one another. In some examples, the elongate arm 24 may rotate to a tilt angle 51 in a first direction, as shown in FIG. 3B, and rotate to the tilt angle 53 in an opposite second direction as shown in FIG. 3C. In some examples, the first and second directions may be about 180 degrees apart and/or the elongate arm 24 may tilt back-and-forth, such that the elongate arm 24 may tilt 90 degrees in a first direction and in an opposite second direction to cover or otherwise move 180 degrees, which may be referred to as a tilt angle or tilt range Ω, shown by FIG. 3D. In some examples, the elongate arm 24 may further tilt from a degree within the tilt angle or title range Ω, such that the elongate arm 24 may tilt 180 degrees in a first direction and in an opposite second direction (from a common point) to cover or otherwise move 360 degrees or within tilt angle or title range Ω′ as illustrated by FIG. 3E. For example, the elongate arm 24 may tilt in the tilt angle or title range Ω, as shown by FIG. 3D, and as the elongate arm 24 is flexible, the elongate arm 24 may further temporarily tilt to any of tilt angle or title range Ω′ in response to contacting a topographic structure. It will be understood that the elongate arm 24 may be positioned to/at any one of 180 (or 360) degree positions within the full 180 (or 360) degree range, as represented by arrows 51, 53 in FIGS. 3B-3C.

As previously described, the guide tool 50 may include a junction (e.g., 56 of FIG. 2) and that includes friction, detents, and/or other mechanical structures use to releasably retain the angular position of the elongate arm 24 at the selected position. The junction may prevent or mitigate movement of the angular position of the elongate arm 24 in response to contact of the elongate arm 24, such as at tip of the elongate arm 24, with a topographic structure of the patient's body. However, as described above, the angular positon may be temporarily changed due to flexing of the elongate arm 24 in response to mechanical pressure caused by contact of the elongate arm 24 with a topographic structure.

In some examples, as previously described, the guide tool 50 includes a mechanism to permit the clinician to control the angular position of the elongate arm 24. The mechanism may be located at the distal portion 23 of the guide tool 50, such as a mechanism on a handle 27 of the guide tool 50 that the clinician may pull (e.g., a wire or other element) to select and/or control a tilt angle and/or rotational angle, and maintain the tilt angle and/or rotational angle of the elongate arm 24.

FIGS. 4A-4E are diagrams schematically representing another example operation of a guide tool. The guide tool 52 of FIGS. 4A-4E may include at least some of substantially the same features and attributes as the guide tool 20 of FIG. 2, the common features and attributes not being re-described below.

FIG. 4A illustrates an example of a first state of the guide tool 52 in which the elongate arm 24 is aligned with the longitudinal axis of the distal elongate portion 26. FIGS. 4B-4C illustrate tilting and rotating of the elongate arm 24. FIGS. 4D-4E illustrate a second state of the guide tool 52 in which the elongate arm 24 is unaligned with the longitudinal axis of the distal elongate portion 26 with elongate arm 24 titled at a tilt angle 51, such as described in FIGS. 3A-3E.

FIG. 4B is a side view of the guide tool showing longitudinal axis A of the distal elongate portion 26 as compared to the longitudinal axis B of the elongate arm 24 while the elongate arm 24 is at a perpendicular tilt angle Ω (e.g., 90 degrees). As shown by the side view, in some examples, the guide tool includes a junction 56 that enables the movement of the elongate arm 24 to the perpendicular tilt angle Ω. The junction 56 may further enable rotation, and may be included in any of the illustrated guide tools, such as those illustrated by FIG. 2, FIGS. 3A-3E, FIGS. 4A and 4C-E, among others. In some examples, the guide tool includes a wire 57 or another mechanism that extends longitudinally through the elongate portion 25 to enable the elongate portion 25 to remain stable (e.g., not rotate) at times, as previously described.

FIG. 4C is end view showing longitudinal axis B of the elongate arm 24 at the perpendicular tilt angle Ω (e.g., 90 degrees) relative to longitudinal axis A of the distal elongate portion 26, and range of degree positions/angular positions (α) of longitudinal axis B of the elongate arm 24 within a full 360 range of motion/rotational angle relative to longitudinal axis A of distal elongate portion 26.

As shown in FIGS. 4D-4E, the elongate arm 24 may rotate about a rotational angle α relative to a longitudinal axis of the distal elongate portion 26 (e.g., 360 degrees), while the elongate arm 24 is at the tilt angle θ, to identify a surface(s) of the vasculature.

A tilt angle (or range) 51, Ω, as illustrated in FIG. 3B, FIG. 4B, and FIG. 4D, includes and/or refers to an angle by which a longitudinal axis B of the elongate arm 24 is tilted with respect to a longitudinal axis A of the distal elongate portion 26 of the guide tool 50, 52, such that the elongate arm 24 is not centrally aligned with (does not share same longitudinal axis as) the distal elongate portion 26. A rotational angle (or range) 54, a relative to the longitudinal axis of the distal elongate portion 26, as illustrated in FIG. 4C and FIG. 4E, includes and/or refers to rotation of the elongate arm 24 about the longitudinal axis A, which may be while the elongate arm 24 is at the tilt angle 51, Ω.

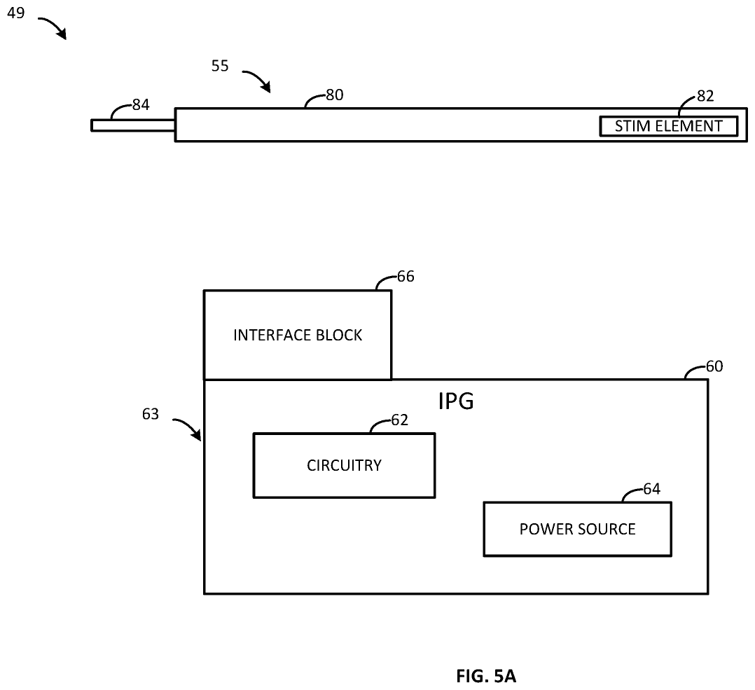

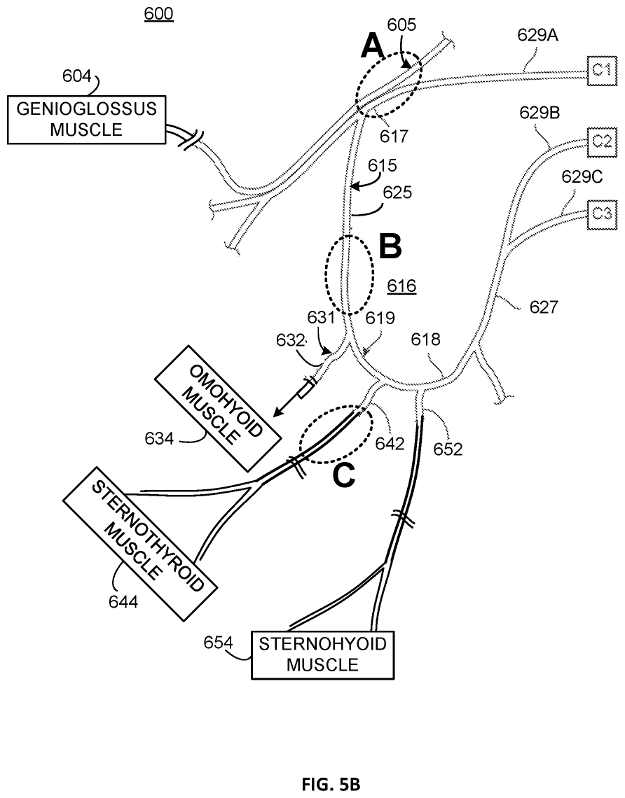

FIG. 5A illustrates an example IMD 49. In some examples, the IMD 49 may include a lead 55 that includes at least some of substantially the same features and attributes as, and/or an example implementation of, the guide tool 20 of FIG. 2. For example, the guide tool may comprise the lead 55 which is connectable to an IPG assembly 63 to form an IMD 49.

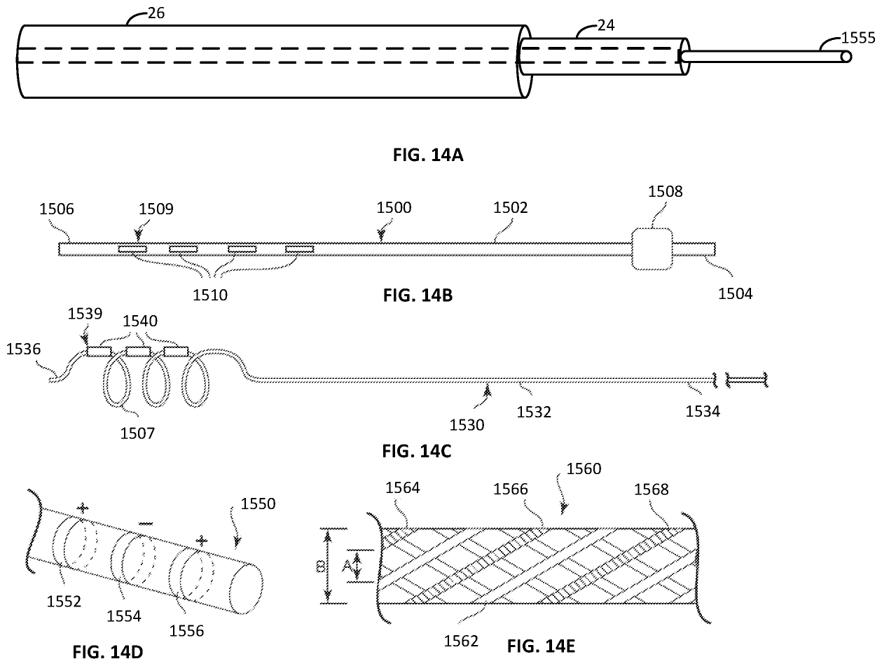

In other examples, the guide tool may be used to guide the lead 55 including the stimulation element 82 through the vasculature. For example, the guide tool may include a guide catheter, with the lead 55 being maneuvered through the guide catheter, as further described later in association with FIG. 14A.

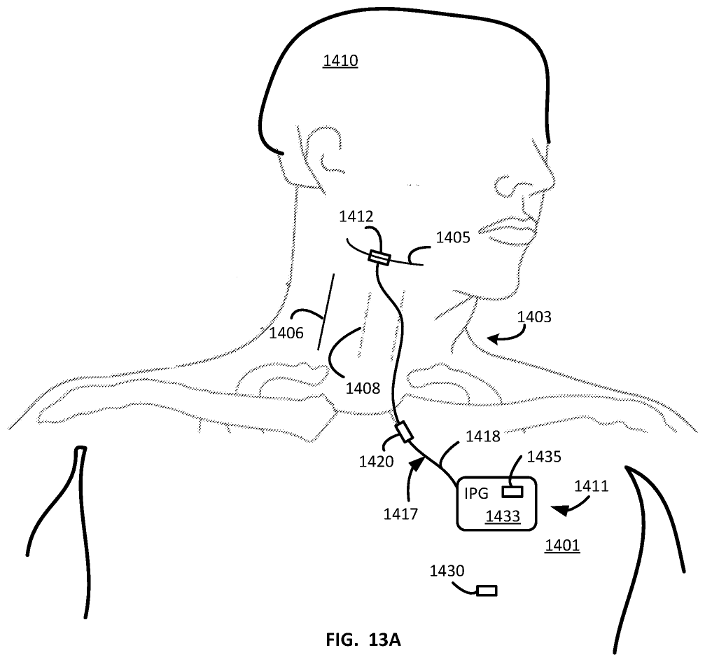

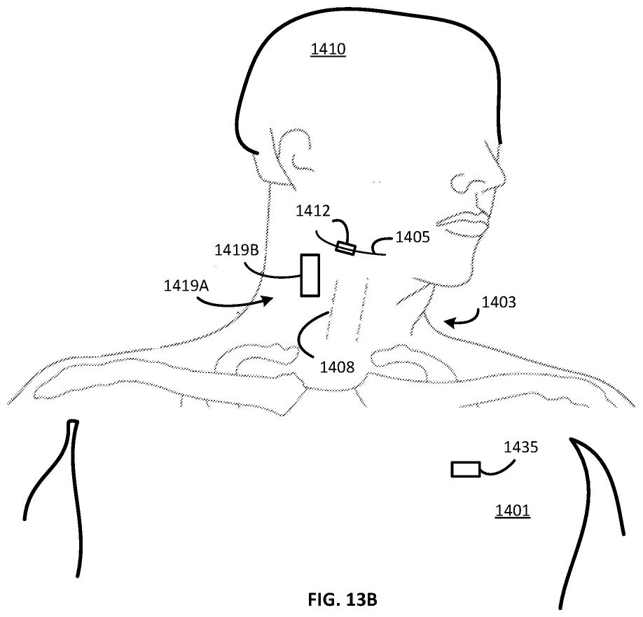

In general terms, the IMD 49 is configured for implantation into a patient, and is configured to provide and/or assist in providing care to the patient. In some examples, the IMD 49 may comprise an IPG for managing sensing and/or stimulation therapy. As further shown in FIG. 13A, the IMD 49 may be chronically implanted in a pectoral region of a patient. Examples are not so limited and the IMD 49 may be implanted in other regions of a patent, such as the head and/or neck of the patient. In some examples, the IPG, as further described below, may be provided in a miniaturized form such as a microstimulator connectable to the lead, with the microstimulator implanted within the head-and-neck region (instead of pectorally) subcutaneously or intravascularly.

As shown in FIG. 5A, the IMD 49 may include an IPG assembly 63 and at least one stimulation lead 55. The IPG assembly 63 may include a housing 60 containing circuitry 62 and a power source 64 (e.g., battery), and an interface block or header-connector 66 carried or formed by the housing 60. The housing 60 is configured to render the IPG assembly 63 appropriate for implanting into a human body, and may incorporate biocompatible materials and hermetic seal(s). The circuitry 62 may include circuitry components and wiring appropriate for generating stimulation signals (e.g., converting energy provided by the power source 64 into a stimulation signal), for example in the form of a stimulation engine. In some examples, the circuitry 62 may include telemetry components for communication with external devices. For example, the circuitry 62 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

As further shown in FIG. 5A, in some examples, the stimulation lead 55 includes a lead body 80 with a distally located stimulation element 82. At an opposite end of the lead body 80, the stimulation lead 55 includes a proximally located plug-in connector 84 which is configured to be removably connectable to the interface block 66. The interface block 66 may include or provide a stimulation port sized and shaped to receive the plug-in connector 84.

The stimulation element 82 may comprise at least one electrode and may include some non-conductive structures biased to (or otherwise configurable to) releasable secure the stimulation element 82 within a vessel relative to a target tissue, among other formats. In some examples, the stimulation element 82 may include an array of electrodes to deliver a stimulation signal to the target tissue. In some non-limiting examples, the stimulation element 82 may comprise at least some of substantially the same features and attributes as described within at least U.S. Pat. No. 9,889,299, issued Feb. 13, 2018, entitled “TRANSVENOUS METHOD OF TREATING SLEEP APNEA” and/or the PCT application published as WO 2021/242633 on Dec. 2, 2021, entitled SINGLE OR MULTIPLE NERVE STIMULATION TO TREAT SLEEP DISORDERED BREATHING, corresponding to U.S. National Stage Application, Ser. No. 17/926,010, filed on Nov. 17, 2022, and published on Jun. 8, 2023 as U.S. Publication 2023/0172479, the teachings of each being hereby incorporated by reference in their entirety.

In some examples, the lead body 80 is a generally flexible elongate member having sufficient resilience to enable advancing and maneuvering the lead body 80 intravascularly (e.g., within the vasculature) to place the stimulation element 82 at a target location adjacent target tissue, such as an upper airway patency-related nerve, such as the hypoglossal nerve and/or the IHM-innervating nerve, etc. In some examples, such as in the case of OSA, the target tissue may include, but is not limited to, the nerve and associated muscles responsible for causing movement of the tongue and related musculature to restore airway patency. In some examples, the target tissue may include, but is not limited to, the hypoglossal nerve and the muscles may include, but is not limited to, the genioglossus muscle. In some examples, the target tissue may include, but is not limited to, the IHM-innervating nerve and its associated innervated muscles which may maintain or increase upper airway patency. In some examples, the hypoglossal nerve and the IHM-innervating nerve may be stimulated in a complementary manner. In some examples, lead body 80 may have a length sufficient to extend from the IPG assembly 63 implanted in one body location (e.g., pectoral) and to the target location (e.g., head, neck). Upon generation via the circuitry 62, a stimulation signal is selectively transmitted to the interface block 66 for delivery via the stimulation lead 55. However, as previously described, in some examples, the lead body 80 may have a length which extends solely within a head-and-neck region, such as when the IPG assembly 63 is configured in a microstimulator size for intravascular placement or subcutaneous placement, such as (but not limited to) the later described example microstimulator 1419B in FIG. 13B. In some examples, other respiratory-related nerves such as the phrenic nerve may be targeted by lead 55 for stimulation.

As previously noted in connection with at least FIG. 1, in some examples, the target tissue may comprise an upper airway patency-related nerve (e.g., motor nerve), which may comprise an IHM-innervating nerve in addition to, or instead of, a hypoglossal nerve.

In some examples, an IHM-innervating nerve may comprise a nerve or nerve branch which innervates (directly or indirectly) at least one infrahyoid muscle (IHM), which may sometimes be referred to as an infrahyoid strap muscle. In some examples, IHM-innervating nerves/nerve branches extend (e.g., originate) from a nerve loop called the ansa cervicalis (AC) or the “AC loop nerve”, which stems from the cervical plexus, e.g., extending from cranial nerves C1-C3. Accordingly, in some examples, at least some IHM-innervating nerves may correspond to an ansa cervicalis (AC)-related nerve in the sense that such nerves/nerve branches (e.g., IHM-innervating nerves) do not form the AC loop nerve but extend from the AC loop nerve. At least because the AC loop nerve is the origin for some nerves which innervate muscles other than the infrahyoid muscles, some AC-related nerves do not comprise IHM-innervating nerves. Moreover, it will be understood that in some examples, stimulation applied to a portion (e.g., superior root) of the AC loop nerve (and/or to nerves from which the AC loop nerve originates) may activate IHM-innervating nerves/nerve branches, which extend from the AC loop nerve. However, implementing stimulation (e.g., to influence upper airway patency) occurring at more proximal locations, such as along the superior root of the AC loop nerve may be more complex because of the number/type of different nerves and number/type of different muscles innervated via a superior root of the AC loop nerve such that selective activation of a particular infrahyoid muscle (via stimulation along the superior root) may be quite challenging in some circumstances.

With this background in mind, FIG. 5B is a diagram 600 schematically representing patient anatomy and providing further details regarding example devices and/or example methods for stimulating an IHM-innervating nerve and/or hypoglossal nerve. The diagram 600 of FIG. 5B includes a side view schematically representing an AC-main nerve 615, in context with a hypoglossal nerve 605 and with cranial nerves C1, C2, C3. As shown in FIG. 5B, portion 629A of the AC-main nerve 615 (e.g., a portion or trunk connecting to the AC loop nerve 619) extends anteriorly from a first cranial nerve C1 and a segment 617 running alongside (e.g., coextensive with) the hypoglossal nerve 605 for a length until the AC-main nerve 615 diverges from the hypoglossal nerve 605 to form a superior root 625 of the AC-main nerve 615, which forms part of the AC loop nerve 619. A portion of the hypoglossal nerve 605 extends distally to innervate the genioglossus muscle 604. As further shown in FIG. 5B, the superior root 625 of the AC-main nerve 615 extends inferiorly (e.g., downward) until reaching near bottom portion 618 of the AC loop nerve 619, from which the AC loop nerve 619 extends superiorly (e.g., upward) to form an lesser root 627 (e.g., inferior root) which joins to the second and third cranial nerves, C2 and C3, respectively and via portions 629B, 629C.

As further shown in FIG. 5B, several branches 631 extend off the AC loop nerve 619, including branch 632 which innervates the omohyoid muscle group 634, branch 642 which innervates the sternothyroid muscle group 644 and at least a portion (e.g., inferior portion) of the sternohyoid muscle group 654. Another branch 652, near bottom portion 618 of the AC loop nerve 619, innervates at least a portion (e.g., superior portion) of the sternohyoid muscle group 654. In some examples, the collective arrangement of the AC-main nerve 615 (including at least superior root 625 of the AC loop nerve 619) and its related branches (e.g., at least 632, 642, 652) when considered together, or any of those elements individually, may sometimes be referred to as an IHM-innervating nerve 616. It will be further understood that at least one such IHM-innervating nerve 616 is present on both sides (e.g., right and left) of the patient's body.

In some examples, stimulation of the superior root 625 of AC loop nerve 619 and/or at least some of the branches 631 extending from the AC loop nerve 619, may influence upper airway patency. However, in some examples, upper airway patency also may be increased and/or maintained by directly stimulating the above-identified muscle groups, such as the omohyoid, sternothyroid, and/or sternohyoid muscle groups. Accordingly, in some examples, such stimulation also may comprise stimulation of just a nerve portion(s), just muscle portion(s), a combination of nerve portion(s) and muscle portion(s), a neuromuscular junction of nerve portion(s) and muscle portion(s), and combinations thereof. Among other effects, in some examples stimulation of such nerves and/or muscles (and/or neuromuscular junctions, combinations, etc.) may act to bring the larynx inferiorly, which may increase upper airway patency.

Stimulation may be delivered to many different locations of an IHM-innervating nerve 616/nerve branches. Of these various potential stimulation locations, FIG. 5B generally illustrates three example stimulation locations A, B, and C, and a stimulation element may be placed at all three of these locations or just some (e.g., one or two) of these example stimulation locations. At each location, a variety of types of stimulation elements (e.g., cuff electrode, axial array, paddle electrode, etc.) may be implanted depending on the particular delivery path, method, etc. As may be appreciated, any of the described stimulation element(s) can be replaced with or include a sensing element in various examples. In some examples, the stimulation element(s) can provide stimulation and/or sensing.

With further reference to FIG. 5B, at each example stimulation A, B, C, a stimulation element may be delivered subcutaneously, intravascularly, etc. At each stimulation location, in some examples the stimulation element may comprise a microstimulator.

It will be understood that these example stimulation locations A, B, C are not limiting and that other portions along the IHM-innervating nerve 616/nerve branches may comprise suitable stimulation locations, depending on the particular objectives of the stimulation therapy, on the available access/delivery issues, etc.

Among the different physiologic effects resulting from stimulation of the various portions of the IHM-innervating nerve 616/nerve branches (and/or innervated muscle portions, neuromuscular junctions, etc.), in some examples stimulation of nerve branches which cause contraction of the sternothyroid muscle 644 and/or the sternohyoid muscle 654 may cause the larynx to be pulled inferiorly, which in turn may increase and/or maintain upper airway patency in at least some patients. Such stimulation may be applied without stimulation of the hypoglossal nerve 605 or may be applied in coordination with stimulation of the hypoglossal nerve 605. More particularly, FIG. 5B show example target tissue including or associated with an IHM-innervating nerve 616 and muscles 634, 644, 654 innervated thereby.

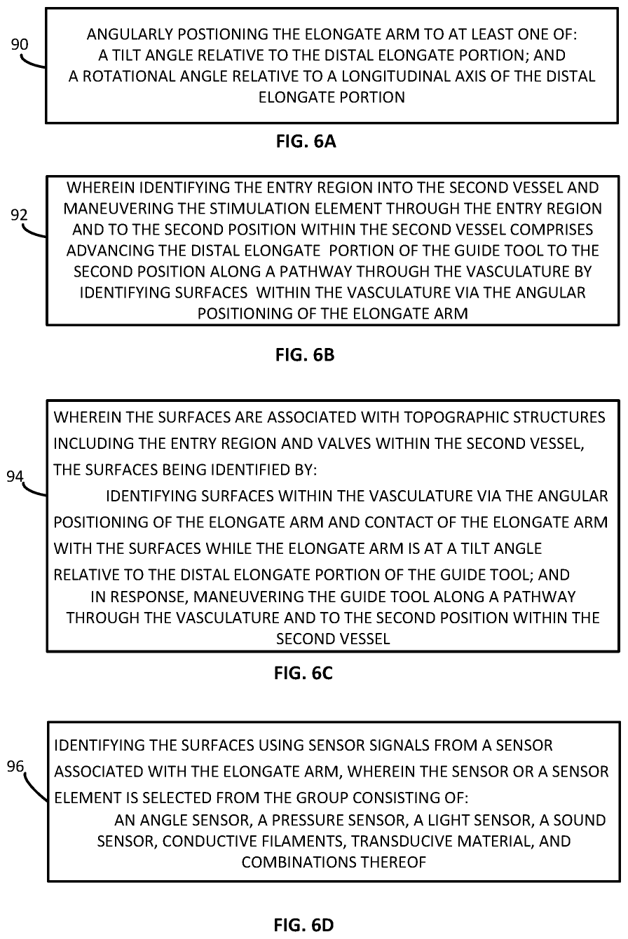

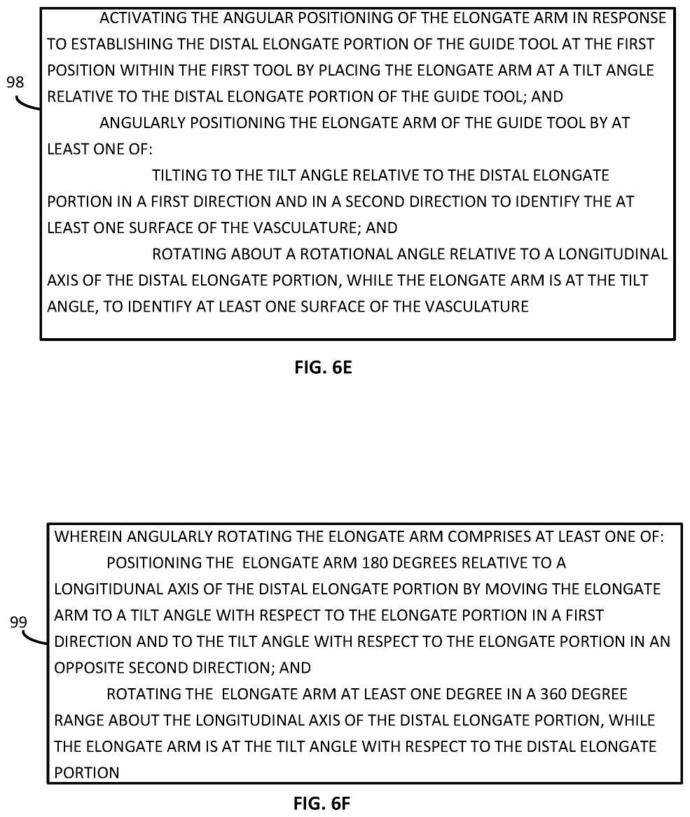

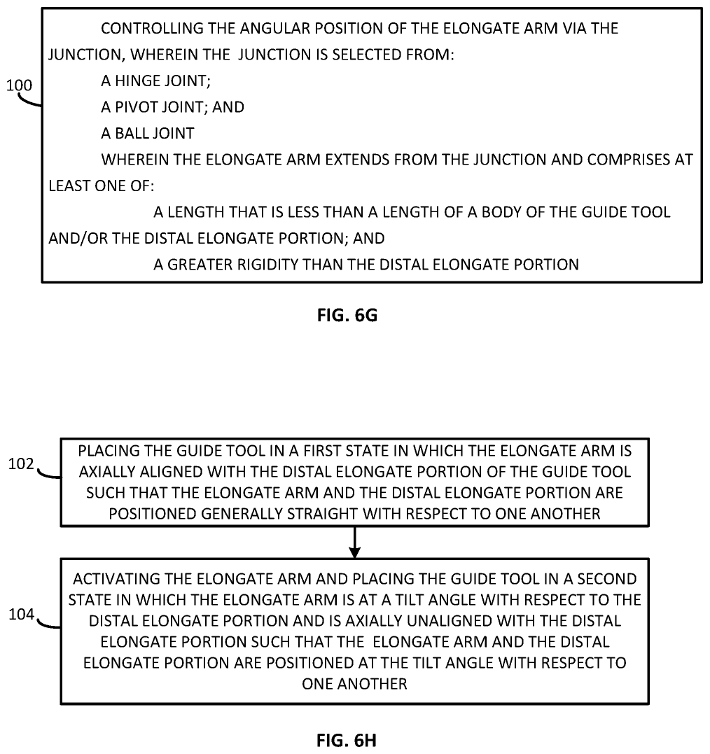

FIGS. 6A-9B are diagrams, which may comprise part and/or are example implementations of a flow diagram in an example method. In some examples, the methods illustrated by FIGS. 6A-9B may comprise part of and/or are example implementations of the method 10 illustrated in FIG. 1. The guide tools illustrated in FIGS. 2 and 3A-4E and/or the IMD illustrated in FIG. 5 may be used to implement the methods illustrated in FIGS. 6A-9B.

As shown at 90 in FIG. 6A, methods, such as method 10 of FIG. 1, may comprise angularly positioning the elongate arm at least one of i) a tilt angle relative to the distal elongate portion of the guide tool, and ii) a rotational angle relative to a longitudinal axis of the distal elongate portion. As previously described, the angular positioning of the elongate arm may accentuate palpation and may be used to identify surfaces within the vasculature, such as entry regions, valves, and/or other topographic structures. For example, referring to FIG. 1, identifying an entry region into the second vessel from the first vessel and maneuvering the stimulation element through the entry region and to the second position within the second vessel, at 14 and 16, may comprise advancing the distal elongate portion of the guide tool to the second position along a pathway through the vasculature by identifying surfaces within the vasculature via the angular positioning of the elongate arm, as shown at 92 in FIG. 6B.

As previously described, the surfaces may include or be associated with topographic structures including the entry region into the second vessel and valves within the second vessel. As shown at 94 in FIG. 6C, the surfaces may be identified by identifying surfaces within the vasculature via the angular positioning of the elongate arm and contact of the elongate arm with the surfaces while the elongate arm is at a tilt angle relative to the distal elongate portion of the guide tool, and, in response, maneuvering the guide tool along a pathway through the vasculature and to the second position within the second vessel. In some examples, this surface identification is accomplished via recognizing differences in palpable feel (as perceived by clinician at a handle of guide tool during maneuvering of the guide tool) among the different topographic structures within the vasculature as a tip of the elongate arm makes contact with such surfaces (and/or passes by a void, such as an entry region) as the tip of the elongate arm moves along the internal surfaces, voids, of the vasculature. In some examples, the entry region is identified and the stimulation element is maneuvered through the entry region and to a second position within the second vessel without anchoring the distal portion (e.g., the elongate arm) of the guide tool at the entry region to the second vessel.

In some examples, the guide tool may include a sensor or a sensor element coupled to a sensor. The sensor may be located on or proximate to the elongate arm. In some examples, the sensor may be located on the distal elongate portion proximate to the elongate arm. For example, as shown at 96 in FIG. 6D, identifying the surfaces may comprise using sensor signals from a sensor or sensor element associated with the elongate arm. In other examples, the sensor may be located on the proximal portion or other portions of the elongate portion, and may be used to sense an amount of force transmitted by the clinician through the length of the guide tool and to assist with accentuating palpation. The sensor or sensor element may be selected from the group consisting of an angle sensor, a pressure sensor, a light sensor, a sound sensor, conductive filaments, transducer material, and combinations thereof, as illustrated in FIGS. 18A-18D.

As shown at 98 in FIG. 6E, methods, such as method 10 of FIG. 1, may comprise activating the angular positioning of the elongate arm in response to establishing the distal elongate portion of the guide tool at the first position within the first vessel by placing the elongate arm at a tilt angle relative to the distal elongate portion of the guide tool. The method 10 may further comprise angularly positioning the elongate arm of the guide tool by at least one of: i) tilting to the tilt angle (e.g., back and forth) relative to the distal elongate portion of the guide tool in a first direction and to a second tilt angle in an opposite second direction to identify the at least one surface of the vasculature; and ii) rotating about a rotational angle relative to a longitudinal axis of the distal elongate portion, while the elongate arm is at the tilt angle, to identify at least one surface of the vasculature, as previously illustrated in FIGS. 3A-4E.

In some examples, activating the angular positioning of the elongate arm comprises at least one of mechanically, electrically, and magnetically causing (e.g., via junction 56) the elongate arm to transition from a first state to a second state associated with an angular position of the elongate arm relative to the distal elongate portion 26. In response to the second state, the method(s) include rotating the elongate arm to the tilt angle relative to the distal elongate portion. The first state and second state are previously illustrated by and described in connection with FIGS. 3A-4E with the first state being associated with the elongate arm being aligned or having a common longitudinal axis with the distal elongate portion and with the second state being associated with the elongate arm being unaligned and having a different longitudinal axis than the distal elongate portion. In some examples, the elongate arm may transition back to the first state in response to palpable pressure by the clinician or sensed pressure, such as in response to pressure from hitting a valve within the second vessel.

In some examples, and referring to FIG. 3A, transitioning back to the first state may provide additional stiffness for pushing through the valve, because in the first state the elongate arm 24 is and/or becomes releasably locked relative to distal elongate portion 26 such that elongate arm 24 and distal elongate portion 26, once again, share the same longitudinal axis. Via this arrangement, pushing force transmitted along/through the distal elongate portion 26 (main shaft of guide tool 50) becomes more directly transmitted through the length of elongate arm 24 and the position of elongate arm 24 may not (or may resist) buckle (at junction 56 of FIG. 4A) relative to the distal elongate portion 26 as the clinician exerts higher pushing force against valve(s) within vessel. It will be understood that, while the elongate arm 24 may be releasably locked relative to distal elongate portion 26 in the first state/position, the distal elongate portion 26 retains previously described steerability by which the combination of the elongate arm 24 and distal elongate portion 26 may be steered as a single unit within vasculature. In other words, the releasable locking of the elongate arm 24 relative to the distal elongate portion 26 (to retain first state for increased pushing capability) does not mean that the elongate arm 24 and distal elongate portion 26 form a rigid structure. Rather, the inherent flexible resilient nature of the arm 24 and distal elongate portion 26 may still be exploited for general maneuvering within/through vasculature.

In some examples, and referring to FIG. 3A, the first and second states of the elongate arm 24 may be used to provide a probing mode and a pushing mode of the guide tool 50. In the probing mode, the elongate arm 24 is in the second state, and the junction (e.g., 56 of FIGS. 2 and 4A) of the guide tool 50 may permit for a greater range of movement of the elongate arm 24, such as tilting and/or rotating. In the pushing mode, the elongate arm 24 is in the first state, and the junction (e.g., 56) of the guide tool 50 may permit for a lesser range of movement of the elongate arm 24 as compared to the pushing mode in order to increase an amount of force that may be transmitted through the tip of the elongate arm 24 for pushing through valves. The different states of the elongate arm 24 may enable the clinician to control the stiffness of the elongate arm 24 so that the elongate arm 24 is less stiff in the probing mode and respectively stiffer in the pushing mode. In some examples, the junction (e.g., 56) may include a mechanical mechanism that telescopically moves the elongate arm 24 to make the elongate arm 24 longer (e.g., less stiff) in the probing mode and shorter (e.g., more stiff) in the pushing mode. In some examples, prior to the distal portion 23 of the guide tool 50 being proximate to the entry region between the first vessel and the second vessel, the elongate arm 24 may be fully retracted in the distal elongate portion 26, to allow for greater flexibility and/or movement through vasculature.

In some examples, as shown at 99 in FIG. 6F, angularly positioning the elongate arm may comprise at least one of: i) positioning the elongate arm 180 degrees relative to a longitudinal axis of the distal elongate portion of the guide tool by moving the elongate arm to a tilt angle with respect to the distal elongate portion in a first direction and to the tilt angle with respect to the distal elongate portion in an opposite second direction, as shown in FIGS. 3A-3E, and ii) rotating the elongate arm to at least one degree position within a 360 degree range about the longitudinal axis of the distal elongate portion, while the elongate arm is at the tilt angle with respect to the distal elongate portion, as shown in FIG. 4A-4E. Examples include other tilt angles and/or rotation angles.

In some examples, the distal elongate portion of the guide tool further comprises a mechanism or junction coupled to the elongate arm and defining a junction between the elongate arm and the distal elongate portion, and, as shown at 100 in FIG. 6G, methods may further comprise controlling the angular position of the elongate arm via the junction. The junction may comprise at least some of substantially the same features and attributes as, or is an example implementation of, junction 56 in FIGS. 2 and 4A. The junction may include a hinge joint to permit tilting of the elongate arm, a pivot joint to permit rotation of the elongate arm, and/or a ball (e.g., ball and socket) joint to permit tilting and rotation of the elongate arm. In some examples, the elongate arm extends from the junction of the guide tool and the elongate arm comprises at least one of a length that is less than a length of the body of the guide tool and/or the distal elongate portion, and a greater rigidity than the distal elongate portion. It will be understood that, depending on the expected particular anatomical application of the guide tool, the implemented rigidity of the elongate arm may be related to (or depend on) the implemented length of the elongate arm and vice versa. Moreover, in some examples, the elongate arm may comprise a rigidity the same as, or less than, a rigidity of the distal elongate portion, depending on the expected anatomical application of the guide tool. In addition, as noted elsewhere herein, the elongate arm may comprise a variable rigidity such as (not limited) to the elongate arm having a greater rigidity in proximal portion of the elongate arm and a less rigidity in the distal portion of the elongate arm.

In some examples, as shown in FIG. 6H, identifying the entry region into the second vessel and maneuvering the stimulation element to the second position within the second vessel, as previously described by method 10 in FIG. 1, may comprise placing the guide tool in a first state, at 102, and activating the elongate arm and placing the guide tool in a second state, at 104. In the first state, the elongate arm is axially aligned with the distal elongate portion of the guide tool such that the elongate arm and the distal elongate portion are positioned generally straight with respect to one another (e.g., general straight state). Sometimes the first state may be referred to as a non-tilted position and/non-tilted angle. In some examples, the elongate arm may be locked in position while in the first state, which may prevent unintended movement of the elongate arm, such as movement caused by contact with topographic structures. In the second state, the elongate arm is at a tilt angle with respect to the distal elongate portion and is axially unaligned with the distal elongate portion such that the elongate arm and the distal elongate portion are positioned at the tilt angle with respect to one another. Sometimes the second state may be referred to as a tilted position and/or angle.

The various methods described herein, including method 10 of FIG. 1, may comprise identifying the entry region into the second vessel by titling the elongate arm to the tilt angle with respect to the distal elongate portion of the guide tool. While at the tilt angle, the methods may include rotating the elongate arm at a rotational angle relative to a longitudinal axis of the distal elongate portion to identify the entry region via contact of the elongate arm with surfaces of the vasculature. In response to the identification, the method(s) include maneuvering the stimulation element through the entry region and to the second position in the second vessel by advancing the distal elongate portion of the guide tool through the entry region and into the second vessel and advancing (e.g., pushing through) the distal elongate portion of the guide tool through the at least one valve and to a target location within the second vessel adjacent or proximate to the target tissue. The entry region and/or valves may be identified by the guide tool accentuating palpation and/or by a sensor of the guide tool, as previously described. In some examples, advancing the distal elongate portion of the guide tool through the at least one valve comprises opening the at least one valve and advancing the elongate arm through the at least one valve via pressure (upon a clinician exerting an axial pushing force through the body of guide tool, including the distal elongate portion, which becomes transmitted through length of elongate arm to its tip) applied by the elongate arm to the at least one valve.

FIGS. 7A-7B are flow diagrams, which may comprise part of (and/or an example implementation of) an example method (e.g., method 10). In some examples, identifying the entry region into the second vessel and maneuvering the stimulation element through the entry region and to the second position within the second vessel, such as illustrated at 14 and 16 of the method 10 in FIG. 1, comprises inserting the lead into the first vessel that connects to the second vessel, at 110, and advancing the lead including the stimulation element to the second position proximate to a target location within the second vessel proximate or adjacent to the target tissue at 112 in FIG. 2A. For example, the lead is advanced into at least the entry region and through a plurality of valves of the second vessel via the elongate arm.

For example, the guide tool may comprise a lead which is connectable to an IPG to form an IMD. In some such examples, the guide tool comprises a lead comprising a proximal portion and the distal portion, with an elongate portion therebetween and including the distal elongate portion, the stimulation element being disposed on or proximate to the distal elongate portion of the lead, and the stimulation element including at least one electrode. In some examples, establishing the stimulation element in the first position within the first vessel of the vasculature which is in communication with the second vessel of the vasculature and maneuvering the stimulation element to the second position within the second vessel comprises inserting the lead within the first vessel of the vasculature, and advancing the distal elongate portion of the lead including the stimulation element to the second position proximate to a target location within the second vessel proximate or adjacent to the target tissue. As shown at 115 in FIG. 7B, the method may further comprise inserting an IPG subcutaneously within a body of the patient and connecting the proximal portion of the lead to the IPG. In some such examples, the connection of the proximal portion of the lead to the IPG may comprise one segment of the proximal portion of the lead extending within the vasculature before passing through a side wall of the vasculature for extension subcutaneously to the IPG.

FIG. 8 is a flow diagram, which may comprise part of a flow diagram in an example method (e.g., method 10). In some examples, the guide tool comprises a guide catheter and a lead is maneuvered through the guide catheter. The guide tool may comprise the guide catheter comprising a proximal portion and the distal portion, with an elongate portion therebetween and including the distal elongate portion. In some examples, as shown in FIG. 8, establishing the stimulation element in the first position within the first vessel of the vasculature which is in communication with the second vessel of the vasculature and maneuvering the stimulation element to the second position within the second vessel comprises inserting the guide catheter within the first vessel of the vasculature, at 120, advancing the distal elongate portion of the guide catheter to a target location within the second vessel proximate or adjacent to the target tissue, at 122, and maneuvering a lead comprising the stimulation element through the guide catheter from the proximal portion through the distal elongate portion and to the second position proximate to the target location within the second vessel, at 124. In some examples, the method (e.g., method 10) further comprises removing the guide catheter.

The methods may further comprise introducing a contrast agent within the vasculature via the guide catheter while and/or after advancing the distal elongate portion of the guide catheter proximate to the target location. In some examples, upon establishing the guide catheter within and through at least some of the valves of the target vessel, the guide catheter provides a pathway for transmission of the contrast agent through at least some of the valves of the vessel that may otherwise not be possible in the absence of the guide catheter because of the highly effective manner in which the valves prevent travel of the contrast agent through the target vessel. In some examples, the stimulation element is maneuvered to the second position without use of a contrast agent and, optionally, without use of an imaging system to detect the contrast agent.

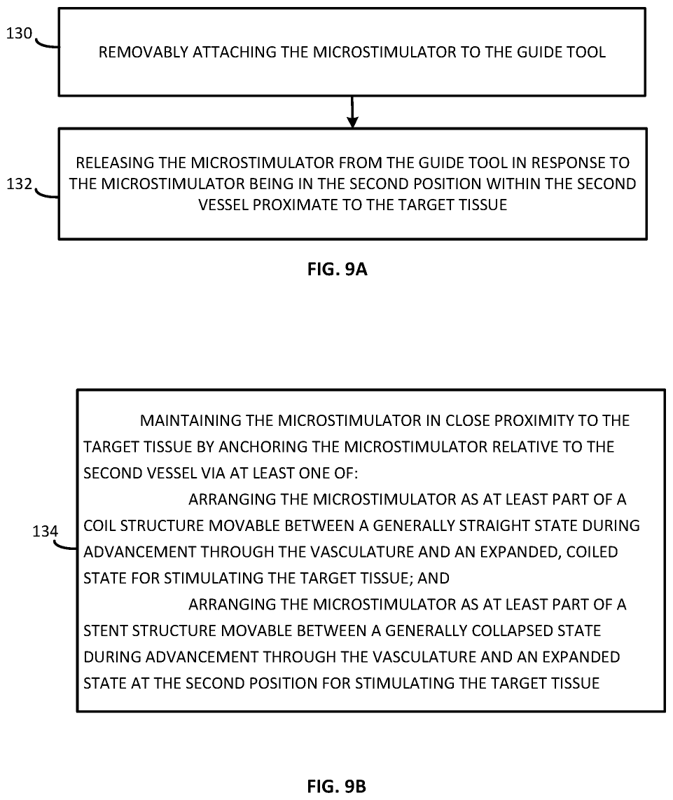

FIGS. 9A-9B are flow diagrams, which may comprise part of a flow diagram in an example method (e.g., method 10). In some examples, the stimulation element comprises a microstimulator comprising at least one electrode. In some examples, as shown in FIG. 9A, maneuvering the stimulation element through the entry region and to the second position comprises at least one of removably attaching the microstimulator to the guide tool, at 130, and releasing the microstimulator from the guide tool in response to the microstimulator being in the second position within the second vessel proximate or adjacent to the target tissue, at 132. The microstimulator may be releasably secured at the distal portion of the guide tool.

In some examples, as shown at 134 in FIG. 9B, methods described herein may comprise maintaining the microstimulator in close proximity to the target tissue by anchoring the microstimulator relative to the second vessel via at least one of: i) arranging the microstimulator as at least part of a coil structure movable between a generally straight state (e.g., uncoiled state) during advancement through the vasculature and an expanded, coiled state for stimulating the target tissue, and ii) arranging the microstimulator as at least part of a stent structure movable between a generally collapsed state during advancement through the vasculature and an expanded state at the second position for stimulating the target tissue, as further illustrated herein by FIGS. 14C and 14E.

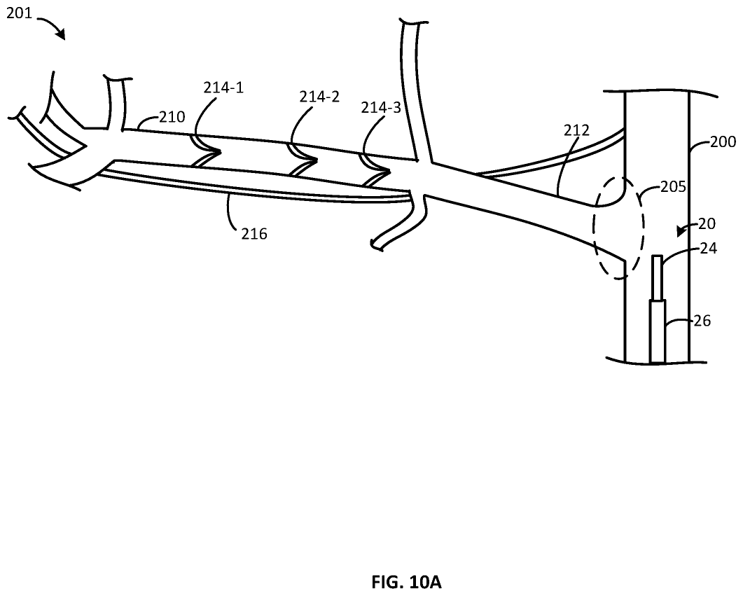

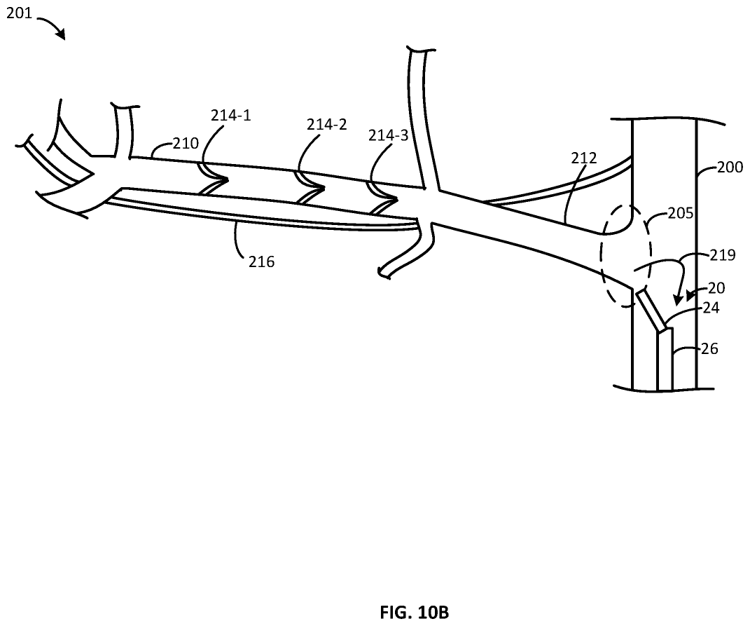

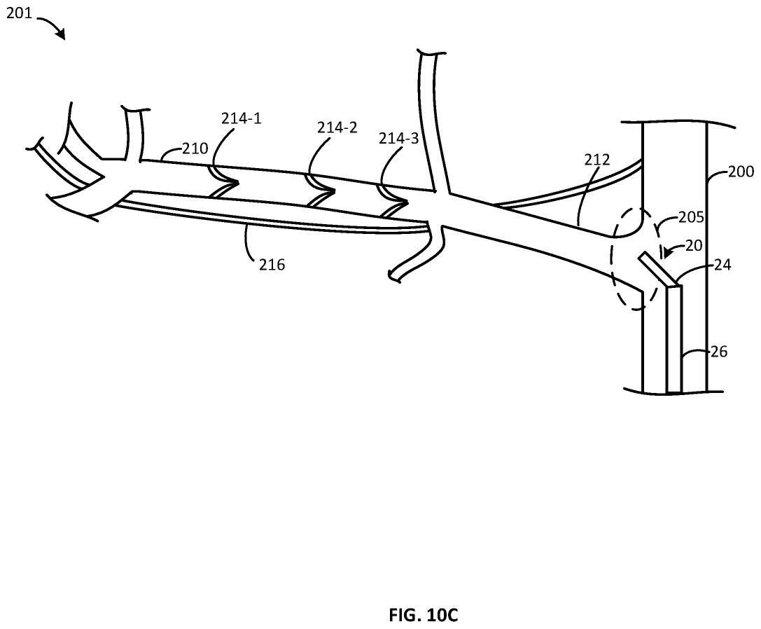

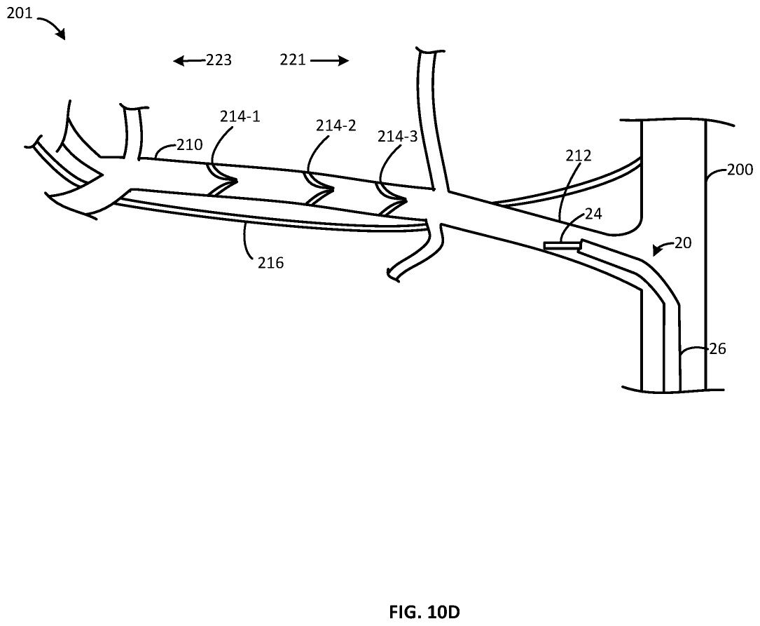

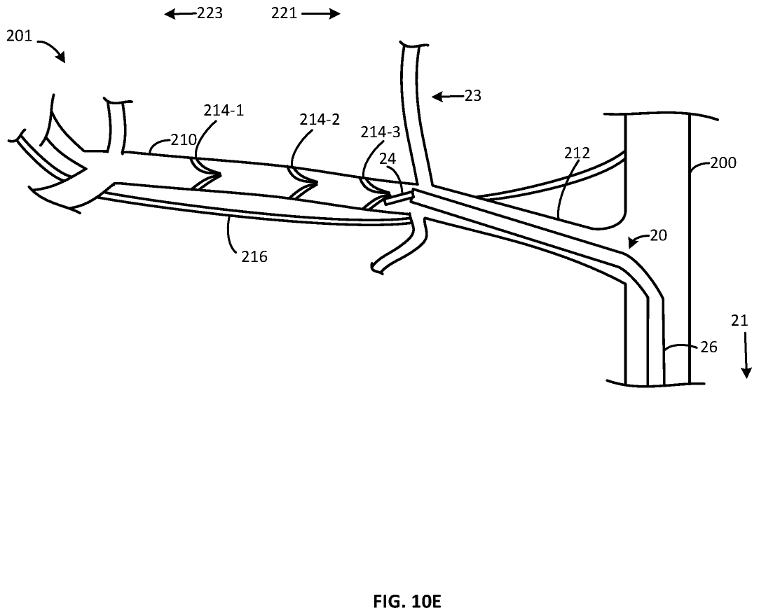

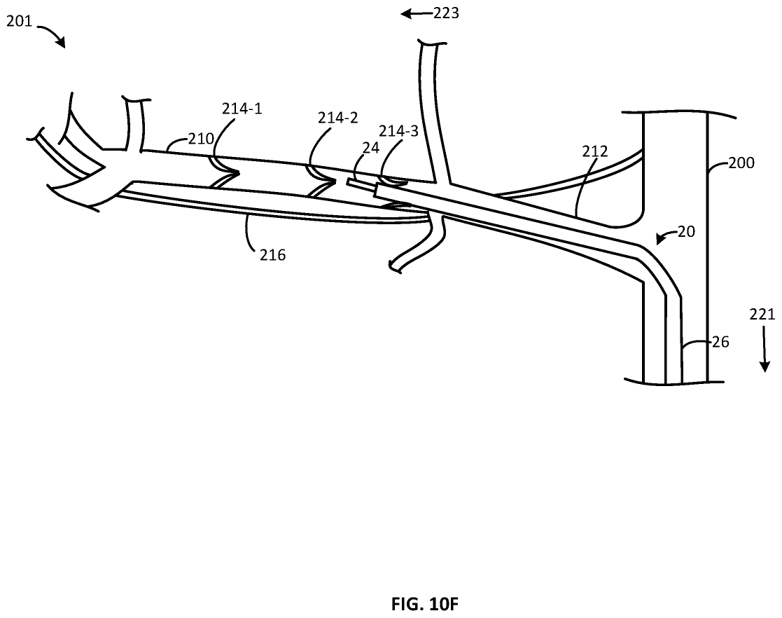

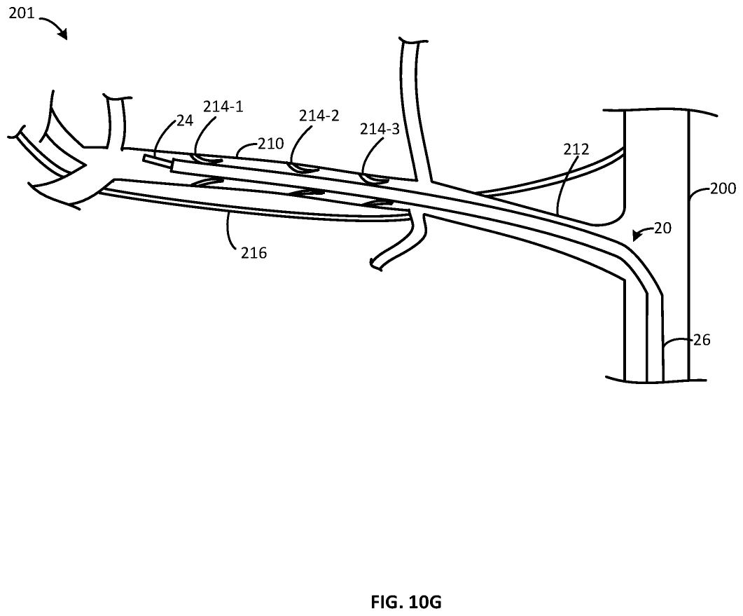

FIGS. 10A-10G are diagrams schematically representing an example of maneuvering a guide tool 20 through a vasculature 201. The guide tool 20 of FIGS. 10A-10G may include at least some of substantially the same features and attributes as any of guide tools 20, 50, 52 of FIGS. 2-4E and/or may be used to implement the method 10 of FIG. 1 and/or FIGS. 6A-9B. The common features and attributes are not re-described below. The guide tool 20 is provided along a pathway through the vasculature 201 toward a location associated with a target tissue 216. The guide tool 20 may include a lead with a stimulation element or may be used to place a stimulation element (which is separate from the guide tool) adjacent to the target tissue 216, in different examples.

As shown in FIG. 10A, the guide tool 20 is inserted into a first vessel 200 and guided toward a first position within the first vessel 200 of the vasculature 201. The guide tool 20 may be in a first state in which the elongate arm 24 is aligned with the longitudinal axis of the distal elongate portion 26. As shown in FIGS. 10B-10C, the guide tool 20 is activated to transition to a second state in which the elongate arm 24 may be angularly positioned (e.g., to a tilt angle 219). The elongate arm 24 may move back-and-forth between a first tilt angle and a second tilt angle, or move to a tilt angle and then rotate about the longitudinal axis of the distal elongate portion 26, as previously illustrated in FIGS. 3A-4E. As shown in FIG. 10B, the elongate arm 24 may periodically contact walls of the first vessel 200, which may be sensed by a sensor and/or through accentuated palpation. As the guide tool 20 is advanced through the vasculature 201, as shown in FIG. 10C, the elongate arm 24 may reach the entry region 205 between the first vessel 200 and the second vessel 210 which is recognized via the above-described accentuated palpation and/or sensing. In some examples, the entry region 205 is associated with a vein trunk 212. As shown in FIGS. 10D-10E, the guide tool 20 is advanced along the vein trunk 212 to the second vessel 210. The second vessel 210 may comprise a plurality of valves 214-1, 214-2, 214-3 which allow fluid flow in a first direction 221 and prevent fluid flow in an opposite second direction 223, with the guide tool 20 being maneuvered in the second direction 223 and against the flow of fluid in the first direction 221. As shown in FIG. 10F, the guide tool 20 pushes through the valve 214-3 by providing pressure on the valve 214-3. In some examples, the guide tool 20 may transition back to the first state illustrated in FIG. 10A to provide additional stiffness for pushing though the valve 214-3. In some examples, upon detecting the presence of a valve (e.g., 214-3), the guide tool 20 may be withdrawn proximally (e.g., in direct shown by 21) a short distance to provide space for transitioning the elongate arm 24 back into the first state prior to once again distally advancing and pushing the elongate arm into and through the valve 214-3. In some examples, the elongate arm 24 may be transitioned back into the first state in response to entering the second vessel 210 from the first vessel 200. The guide tool 20 may continue to be advanced along the second vessel 210 until reaching the second position which is proximate or adjacent to the target tissue 216 (e.g., nerve) to be stimulated, as shown in FIG. 10G.

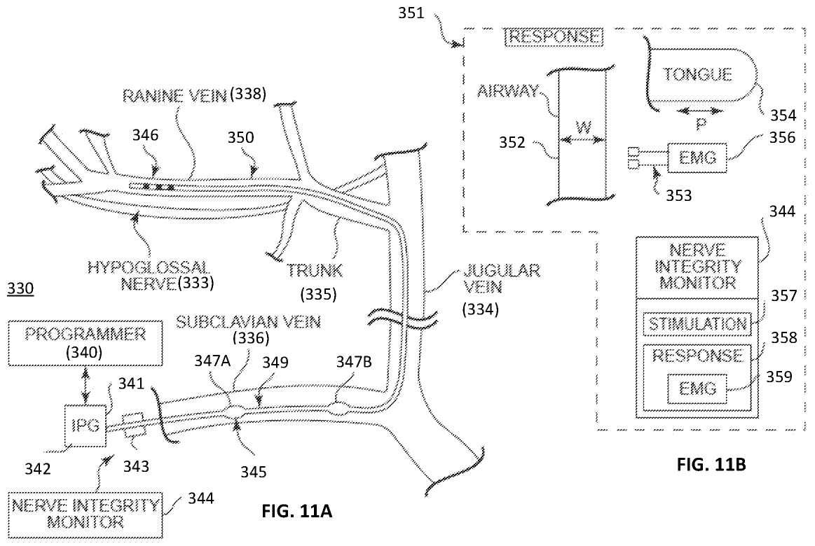

FIGS. 11A-11B are schematic illustrations of vasculature placement of a stimulation element of an IMD. The IMD may include a lead 350 configured to be delivered intravascularly and to be positioned within the vasculature 330. Lead 350 is configured to place a stimulation element 346 within the ranine vein (VCH) 338 adjacent the hypoglossal nerve 333 (or another vessel adjacent the hypoglossal nerve 333 or other target tissue). As previously described, the VCH is sometimes referred to as the ranine vein, which begins at its distal end at a point below the front of the tongue, travels along the distal portion of the hypoglossal nerve 333, and then may join the lingual vein, and eventually opens into the internal jugular vein. In some examples, other veins, such as another branch of the lingual vein may also be a candidate for the stimulation element 346 placement instead of or in addition to the VCH.

In some examples, the lead 350 is used as the guide tool in a method of intravascularly delivering a stimulation element 346 to stimulate a target tissue. In this method, as illustrated in FIG. 11A, lead 350 is introduced into and through the subclavian vein 336 and then is advanced through the jugular vein 334, through vein trunk 335, and into the VCH 338 until stimulation element 346 is within a target position of the VCH 338 (or another vein adjacent the hypoglossal nerve 333).

In some examples, the stimulation signal is applied at a single stimulation site along the hypoglossal nerve 333 or another target tissue. In other examples, as illustrated in and referring to FIG. 12A, the stimulation signal of a SDB therapy is applied from one or more of multiple locations 760, 762, 764, 766, 767, 768, (represented by the symbol x) within one or more vessels to target multiple stimulation sites 770P, 770M, 770D along a target nerve 770, as illustrated in FIG. 12A. The electric field applied at each location is represented schematically by the arrow extending from the symbol x toward the stimulation site 770M, 770P, 770D on target nerve 770. In some examples, these multiple sites include multiple stimulation locations arranged proximally (e.g., 762), midway (e.g., 764), and distally (e.g., 760) within vein 763 along the (hypoglossal) nerve 770, one or more stimulation sites on both the right and left hypoglossal nerves, and/or multiple stimulation sites (proximal 768, midportion 766, and distal 767) along another vein 765 adjacent to the hypoglossal nerve 770. While FIG. 12A depicts three stimulation sites 770P, 770M, 770P on the target nerve 770, examples of the present disclosure may stimulate target nerve 770 at any point (or multiple points) between (or distally beyond or proximally beyond) the identified sites 770P, 770M, 770P along the target nerve 770.

As illustrated in FIG. 12B, in some examples, an IMD 771 includes a lead system 775 comprising two or more stimulation leads 776, 777 that extend from an IPG 742 to enable the leads 776, 777 that extend down each of the respective different intravascular pathways to enable two or more independent stimulation sites on a single target nerve or other tissue from different vessels, such as vein 763 and 765 (FIG. 12A). Each lead 776, 777 may include one, two, or more different electrode portions 780, 782, 784 spaced apart from each other along a length of the distal portion 779 of each lead 776, 777, as illustrated in FIG. 12B. In some examples, the electrode portions 780, 782, 784 of each lead 776, 777 are arranged with a minimum distance (D1 or D2) therebetween such that the stimulation signal applied at one electrode portion is separate and independent from the stimulation signal applied at the other electrode portions to achieve independent stimulation sites along the same target tissue. The electrode portions of a distal portion of one lead are spaced apart such that when a stimulation signal from a first electrode portion is applied at one site, the other respective sites are not stimulated by the first electrode portion. In some examples, each of the electrode portions 780, 782, 784 may be activated simultaneously to apply a stimulation signal to each of the spaced apart, independent stimulation sites.

In some examples, the spacing D1 and D2 between the electrodes on the first lead 776 is equal to each other and the spacing D3 and D4 between the electrodes on the second lead 777 is equal to each other, and in other examples, the spacing is substantially different from each other. In some examples, the spacing (D1, D2) between the electrodes on the first lead 776 is the same as the spacing (D3, D4) between the respective electrode portions on the second lead 777. In other examples, the spacing (D1, D2) between the electrodes on the first lead 776 are the different than the spacing (D3, D4) between the electrode portions on the second lead 777 to account for the different distances traveled intravascularly by the respective leads 776, 777 to locate the different respective electrode portions at the stimulation sites.

In some examples, the stimulation sites along one or more target tissues are spaced apart from each other by a distance that uses multiple stimulation signals to enable capturing the corresponding portion of the target tissue but wherein the spacing between adjacent stimulation sites along the tissue is close enough to allow overlap between the adjacent stimulation signals. In this way, various stimulation vectors may be applied to enhance capture and suprathreshold stimulation of the target nerve(s).

In some examples, the separate leads 776, 777 of the lead system 775 are positioned within different vessels (e.g., 763, 765 or a different set of veins) to stimulation different tissue. In such examples, one lead 776 stimulates a first nerve (e.g., nerve 770) and the other lead 777 stimulates a second nerve (not shown). In some examples, each of the first and second nerves are associated with control of the respiratory system such that their selective stimulation relative to a respiratory pattern restores and maintains airway patency to alleviate obstructive SDB.

Referring back to FIG. 11A, in some examples, a nerve integrity monitor (standalone monitor 344 or integrated into a SDB physician programmer 340) is used to aid the physician in placing the stimulation element 346 of the lead 350 in the proper location. The nerve integrity monitor may comprise at least some of substantially the same features and attributes as the nerve integrity monitor described in U.S. Pat. No. 6,334,068, entitled INTRAOPERATIVE NEUROELECTROPHYSIOLOGICAL MONITOR, issued on Dec. 25, 2001, and which is hereby incorporated by reference in its entirety. In other examples, other nerve integrity monitors or an equivalent array of instruments (e.g., a stimulation probe and electromyography system) are used to apply the stimulation signal and evaluate the response of the muscle innervated by the target tissue.

In some examples, as shown in FIG. 11B, the nerve integrity monitor 344 comprises a stimulation module 357 and a response module 358 that includes electromyography monitoring electronics (EMG) 359.

In some examples, the vessel and target tissue may include various types of vessels and/or tissue. In some examples, the target tissue may include respiratory-related tissue, such as nerves and/or muscles, muscles innervated by respiratory tissue-related nerves, and/or nerves whose stimulation elicit (via the central nervous system (CNS)) respiratory responses (e.g., reflex opening response).

Non-limiting examples of respiratory-related tissue include an upper airway patency-related tissue (e.g., a hypoglossal nerve, an IHM-innervating nerve, and/or muscles innervated by such nerves), an upper airway reflex-related sensory nerve, and/or a phrenic nerve (and/or diaphragmatic tissue), as well as other nerves and/or other muscles. Example upper airway patency-related muscles may include, but are not limited to, the genioglossus muscle, such as protrusor muscles and IHMs. In some such examples, the hypoglossal nerve may be stimulated at a location and/or manner to activate at least (or solely) the protrusor muscles of the genioglossus muscle. The nerves whose stimulation elicit (via the CNS) respiratory responses may comprise, some examples, the iSL nerve and/or the glossopharyngeal nerves at or near the upper airway.

Some example muscles may comprise diaphragm muscles innervated by the phrenic nerve, among other muscles. Some example muscles also may comprise muscles (and their innervating nerves) which may be activated upon stimulation of upper airway reflex-related sensory nerves (e.g., iSL nerve, glossopharyngeal nerve), which when stimulated, may elicit (via the CNS) a reflex opening response which activates nerves (and their innervated muscles) to facilitate respiration to prevent and/or overcome sleep disordered breathing.

Accordingly, in some examples, the target tissue may comprise upper airway patency-related motor nerves and muscles, which may comprise a hypoglossal nerve, IHM-innervating nerve, and/or other nerves and the muscles innervated by the aforementioned example nerves, associated neuromuscular junctions, etc. In some examples, upper airway patency-related motor nerves may include nerves that stimulate muscles associated with increasing, restoring, or maintaining upper airway patency to promote respiration. In some examples, the target tissue may comprise nerves, muscles, etc. not directly related to upper airway patency, such as the phrenic nerve, diaphragm, or other nerves/muscles relating to respiration. In such examples, the target tissues may comprise the phrenic nerve and/or the diaphragm muscles. In some examples, an IHM-innervating nerve may comprise a nerve or nerve branch which innervates (directly or indirectly) at least one IHM, which may sometimes be referred to as an infrahyoid strap muscle. The IHMs may include the sternohyoid muscle, sternothyroid muscle, thyrohyoid muscle, and omohyoid muscle, as previously noted. In some such examples, the nerve to be stimulated comprises a nerve branch extending from the AC loop nerve which innervates solely the sternothyroid muscle.

In some examples, the target tissues may comprise nerves, which when stimulated, elicit (via the CNS) a response which activates at least some of the above-identified nerves and/or muscles to facilitate respiration to prevent and/or overcome sleep disordered breathing, which are sometimes herein referred to as “upper airway reflex-related sensory nerves”. At least some example upper airway reflex-related sensory nerves include the iSL nerve and the glossopharyngeal nerve. In some examples, stimulation of the hypoglossal nerve may result in protrusion of the tongue (e.g., genioglossus muscle), stimulation of the IHM-innervating nerve may result in contraction of other upper airway muscles. In some such examples, such stimulation may maintain and/or increase upper airway patency to treat at least obstructive sleep apnea.

In some examples, the target tissue to be stimulated may comprise target tissues relating to urination, defecation, etc., and in particular urinary incontinence and/or fecal incontinence such as, but not limited to, stress incontinence. In some such examples, the target tissue may comprise nerves and muscles associated with voiding and/or prevention of voiding, with such nerves and/or muscles being associated with at least the external urinary sphincter and/or external anal sphincter. Among other examples, at least the pudendal nerve comprises one target tissue innervating such muscles, with the target including the pudendal nerve trunk, the deep perineal branch, and/or other portions of the pudendal nerve. At least some further examples of target tissues may comprise the hypogastric nerve and/or pelvic splanchnic nerve. In some examples, the target tissue may comprise nerves, muscles, etc. not directly related to incontinence, such as other nerves/muscles relating to pelvic dysfunction. In some examples, stimulation of at least a portion of the pelvic function-related nerve resulting in contraction of a respective one of the sphincter muscles and/or relaxation of a respective one of the sphincter muscles, or stimulation of the pertinent nerve resulting in contraction (or relaxation) of other pelvic muscles. In some such examples, such stimulation may be delivered to treat at least urinary incontinence and/or fecal incontinence such as, but not limited to, stress incontinence.

FIG. 11B further illustrates a response evaluation array 351. As shown in FIG. 11B, response evaluation array 351 provides mechanisms to evaluate the effectiveness of a target site for stimulating target tissue. In some examples, the array 351 includes: (1) observing or measuring the extent and location (an extension of the base of the tongue is preferred over extension of the tip) of tongue motor response 354, such as but not limited to tongue protrusion (indicated by arrow P); (2) observing or measuring the extent of increased cross-sectional area (indicated by arrow W) of an upper respiratory airway 352; (3) measuring the extent of an EMG response 356 (measured via EMG electronics 359 of monitor 344) of one or more muscles upon stimulation applied at a potential target location within a vessel; (4) observing or detecting a twitch of the tongue or laryngeal muscle; and/or (5) a substantial reduction in apnea events.

The monitor 344 and aspects of the response array 351 may be used to evaluate the positioning of a lead 350 within a vessel relative to a potential stimulation site on a target tissue. In some examples, a repetitive stimulation pattern is applied from the stimulation module 357 of nerve integrity monitor 344 to the stimulation element 346 of the lead 350 as the lead 350 is advanced distally during navigation of the ranine vein (VCH) 338 (FIG. 11A). In some examples, the applied stimulation pattern is a 1 second burst of stimulation every 3 seconds, a ramping stimulation pattern, and/or a physician controlled burst. In other examples, EMG monitoring electronics 359 of the nerve integrity monitor 344 enables measuring a muscle response to the nerve stimulation applied during navigation of the target vessels. Fine wire electrodes 353 (or similar) may be connected in electrical communication with the nerve integrity monitor 344 and are used to continuously monitor the muscle activity in response to the stimulation patterns applied via stimulation element 346 during navigation of the lead 350. Using this arrangement, a closed loop feedback allows the physician to obtain real-time feedback of a position (along the pathway) of the stimulation element 346 and feedback regarding the ability of the stimulation element 346 to capture the target tissue at a particular position of the stimulation elements 346 along the pathway adjacent the target tissue.

Referring again to FIG. 11A, lead 350 includes a lead body 349 that supports a respiratory sensor 345 (including first and second electrodes 347A, 347B) at a proximal portion of lead body 349. In such examples, the respiratory sensor 345 is provided on the same lead body 349 as the stimulation element 346 so that both the respiratory sensor 345 and the stimulation element 346 are placed in the vasculature 330 in a single pass. As the stimulation element 346 is advanced distally for placement adjacent a target tissue, the respiratory sensor 345 may be automatically placed within a pectoral region of the patient for sensing the respiration pattern of the thorax of the patient. With this placement, the sensor 345 detects respiratory features and/or patterns (e.g., inspiration, expiration, respiratory pause, etc.) to trigger activation of stimulation element 346 to stimulate target tissue. With this arrangement, the IPG 341 receives sensor waveforms from the respiratory sensor 345, such that the IPG 341 may deliver electrical stimulation synchronously with inspiration, e.g., with each respiratory breath (or another aspect of the respiratory pattern) according to a therapeutic treatment plan. The respiratory sensor 345 may be powered by IPG 341 and IPG 341 contains internal circuitry to accept and process the impedance signal from the lead 350.

In some examples, a respiratory waveform is monitored and stimulation (generally synchronous with respiration) is not applied until a respiratory feature and/or pattern indicative of an SDB event is identified. Stimulation is terminated upon detection that the feature or pattern is no longer present within the monitored respiratory waveform.

In some examples, the respiratory sensor 345 is an impedance sensor. The impedance sensor may be configured to sense a bio-impedance signal or pattern whereby the control unit evaluates respiratory patterns within the bio-impedance signal. For bio-impedance sensing, electric current may be injected through electrode 347B and an electrically conductive portion of case 342 of the IPG 341 and voltage will be sensed between electrodes 347A and 347B (and/or between 347A and the case 342 of IPG 341) to compute the impedance.

In some examples of bio-impedance sensing, during the placement of the impedance sensing lead, the impedance waveform may be displayed on the programmer 340 in real time. The location of electrodes 347A and 347B may be interactively (an array of electrodes available to select from via a multiplexer switch within the IPG 341) adjusted for optimal signal to noise ratio.

In some examples, the sensor 345 is a pressure sensor. The pressure sensor may detect pressure in the thorax of the patient. In other examples, pressure may be a combination of thoracic pressure and cardiac pressure (e.g., blood flow). With this configuration, a controller is configured to analyze this pressure sensing information to detect the respiratory patterns of the patient.

In some examples, the sensor 345 comprises an accelerometer to sense respiratory information including respiratory effort, respiratory morphology, inspiratory and expiratory phases, etc. In some such examples, the accelerometer-based sensor 345 may comprise at least some of substantially the same features and attributes as described in at least: U.S. Pat. No. 11,324,950 granted on May 10, 2022, and entitled ACCELEROMETER-BASED SENSING FOR SLEEP DISORDERED BREATHING (SDB) CARE; PCT Publication WO 2021/016562 published on Jan. 28, 2021 and entitled RESPIRATION DETECTION, with corresponding U.S. National Stage application Ser. No. 16/977,664, filed Sep. 2, 2020, published on Apr. 20, 2032 as U.S. Publication 2023/0119173; and PCT Publication WO 2021/016558 published on Jan. 28, 2021 and entitled SLEEP DETECTION FOR SLEEP DISORDERED BREATHING (SDB) CARE, corresponding to U.S. National Stage application Ser. No. 16/978,470, filed Sep. 4, 2020, and published on Mar. 30, 2023 as U.S. Publication 2023/0095780 each of which are incorporated herein in their entirety.

In some examples, the lead 350 includes an anchor 343 that is locatable at a proximal portion of lead body 349. The anchor 343 may to ensure the sensor 345 and stimulation element 346 remain in the proper position within the vasculature 330.

At least some of the previously introduced FIGS. 1-12B depict a stimulation element intravascularly delivered into the VCH 338 for stimulating the hypoglossal nerve to treat SDB. In some examples, a method of treating SDB includes identifying a target location to locate stimulation element 346 (FIG. 11A) along a length of the VCH 338 (or another vein suitable to apply stimulation to the hypoglossal nerve or another target tissue) that results in a stimulation of the hypoglossal nerve. At least some of such examples may be implemented using at least some of substantially the same features and attributes as described in U.S. Pat. No. 9,889,299, issued Feb. 13, 2018, entitled “TRANSVENOUS METHOD OF TREATING SLEEP APNEA”, and which is hereby incorporated by reference in its entirety.

FIGS. 13A-13B illustrate example devices that comprise IMDs. The IMDs may include a lead system including at least one lead. The lead(s) of the IMDs may be an implementation of a guide tool and/or may be delivered to a target location using a guide tool, as previously described.

FIG. 13A is a diagram including a front view of an example IMD 1411 implanted within a patient's body 1410. The IMD 1411 may comprise (but is not limited to) an IPG 1433 including a sensor 1435. The IPG 1433 is capable of being surgically positioned within a pectoral region 1401 of a patient 1410, and a stimulation lead 1417 is electrically coupled with the IPG 1433 via a connector (not shown) positioned within a connection port of the IPG 1433. In some examples, the sensor 1435 may comprise an accelerometer sensor, as previously described in some examples of the present disclosure.

The lead 1417 includes a lead body 1418 for chronic implantation (e.g., subcutaneously via tunneling or other techniques) and to extend to a position adjacent a nerve (e.g., hypoglossal nerve 1405 and/or other nerve 1406) or other target tissue. The lead 1417 may comprise a stimulation element, such as the illustrated stimulation electrode 1412, to engage the nerve (e.g., 1405, 1406) in a head-and-neck region 1403 for stimulating the nerve to treat a physiologic condition, such as SDB like OSA. The stimulation electrode 1412 is positioned within a portion of the vasculature adjacent a target nerve, such as the hypoglossal nerve 1405 of the patient 1410, to enable stimulation of the nerve 1405, as described below in detail. In some examples, nerve 1406 may comprise an IHM-innervating nerve for also maintaining or increasing upper airway patency separate from, and/or in a complementary manner with stimulation of the hypoglossal nerve. In some such examples, stimulation of the IHM-innervating nerve and/or the hypoglossal nerve (and/or other nerves) may be performed via at least some of substantially the same features and attributes as described in at least the PCT application published as WO 2021/242633 on Dec. 2, 2021, entitled SINGLE OR MULTIPLE NERVE STIMULATION TO TREAT SLEEP DISORDERED BREATHING, corresponding to U.S. National Stage Application, Ser. No. 17/926,010, filed on Nov. 17, 2022, and published on Jun. 8, 2023 as U.S. Publication 2023/0172479, being hereby incorporated by reference in its entirety.

In some examples, nerve 1406 may comprise a phrenic nerve with stimulation of nerve 1406 to stimulate the diaphragm, such as for treating at least central sleep apnea (CSA).

As described above, the stimulation electrode 1412 may be delivered intravascularly in accordance with at least some of substantially the same features and attributes of method 10 in FIG. 1 and/or using a guide tool described herein. In some examples, the lead 1417 includes the guide tool.

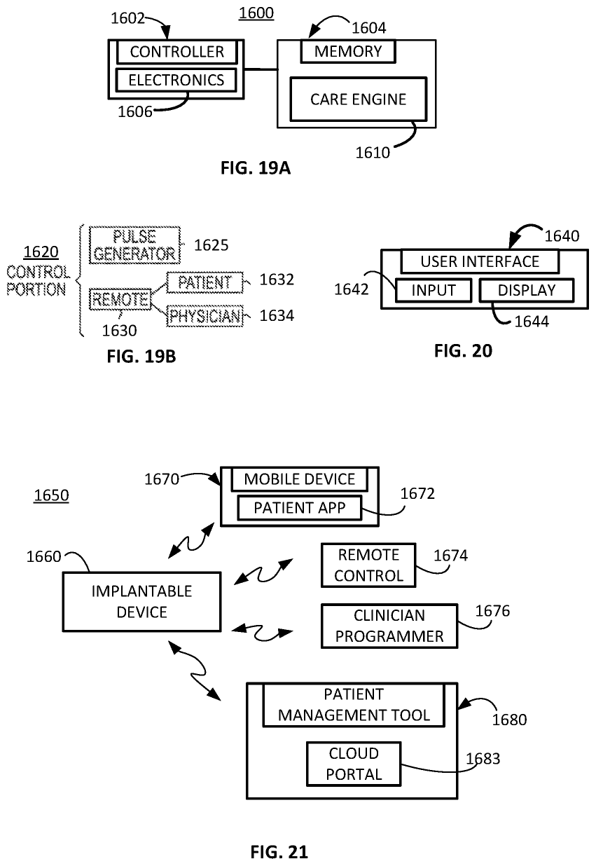

The IMD 1411 may comprise circuitry, power element, etc. to support control and operation of the sensor 1435 and the stimulation electrode 1412 (via lead 1417). In some examples, such control, operation, etc. may be implemented, at least in part, via a control portion (and related functions, portions, elements, engines, parameters, etc.) such as described in association with FIGS. 19A-21.

In various examples, delivering stimulation to an upper airway patency nerve (e.g., a hypoglossal nerve 1405) via the stimulation electrode 1412 is to cause contraction of upper airway patency-related muscles, which may cause or maintain opening of the upper airway (1408) to prevent and/or treat OSA.

In some examples, such electrical stimulation may be applied to an IHM-innervating nerve 1406 via the stimulation electrode 1412 to cause contraction of upper airway patency-related muscles to maintain or increase upper airway patency.

In some examples, separate stimulation leads 1417 may be provided or a single stimulation lead 1417 may be provided but with a bifurcated distal portion with each separate distal portion extending to a respective one of the hypoglossal nerve 1405 and the nerve 1406 (e.g., IHM-innervating nerve, in some examples).

In some examples, the contraction of the hypoglossal nerve and/or contraction of other nerves (e.g., IHM-innervating nerve, in some examples) caused by electrical stimulation comprises a suprathreshold stimulation, which is in contrast to a subthreshold stimulation (e.g., mere tone) of such muscles. A suprathreshold intensity level may correspond to a stimulation energy greater than the nerve excitation threshold, such that the suprathreshold stimulation may provide for higher degrees (e.g., maximum, other) of upper-airway clearance (e.g., patency) and therapy efficacy.

In some examples, a target intensity level of stimulation energy is selected, determined, implemented, etc. without regard to intentionally establishing a discomfort threshold of the patient. Stated differently, in at least some examples, a target intensity level of stimulation is implemented to provide the target efficacious therapeutic effect in reducing SDB without attempting to adjust or increase the target intensity level according to (or relative to) a discomfort threshold.

Some non-limiting examples of such devices and methods to recognize and detect the various features and patterns associated with respiratory effort and flow limitations include, but are not limited to: U.S. Pat. No. 8,938,299, issued Jan. 20, 2015, and entitled “SYSTEM FOR TREATING SLEEP DISORDERED BREATHING”, the entire teachings are hereby incorporated by reference herein in its entirety.

In some examples, various stimulation methods may be applied to treat OSA, which include but are not limited to: U.S. Pat. No. 10,583,297, issued Mar. 10, 2020, and entitled “METHOD AND SYSTEM FOR APPLYING STIMULATION IN TREATING SLEEP DISORDERED BREATHING”; U.S. Pat. No. 8,938,299, issued Jan. 20, 2015, and entitled “SYSTEM FOR TREATING SLEEP DISORDERED BREATHING”; U.S. Pat. No. 5,944,680, issued Aug. 31, 1999, and entitled “RESPIRATORY EFFORT DETECTION METHOD AND APPARATUS”; and U.S. Pat. No. 10,898,703, issued Jan. 26, 2021, and entitled “STIMULATION FOR TREATING SLEEP DISORDERED BREATHING”, the entire teachings of each are hereby incorporated by reference herein in their entireties.

As further shown in FIG. 13A, some examples IMDs may be implemented with additional sensors 1420, 1430 to sense additional physiologic data, such as but not limited to, further respiratory information via sensing transthoracic bio-impedance, pressure sensing, etc. in order to complement the respiration information sensed via an acceleration sensor. In some examples, one or both of the sensors 1420, 1430 may comprise sensor electrodes. In some examples, stimulation electrode 1412 also may act as a sensing electrode. In some examples, at least a portion the IPG 1433 may comprise a sensor or at least an electrically conductive portion (e.g., electrode) to work in cooperation with sensing electrodes to implement at least some sensing arrangements.

Examples are not limited to SDB and may be directed to other neurostimulation devices and cardiac care devices which may detect cardiac signals and provide atrial chamber stimulation therapy. For example, the IMD may include or be coupled to implantable leads using to sense left and right atrial and ventricular cardiac signals. The electronics assembly of the IMD processes the cardiac signals and provides stimulation signals using an IPG and the leads. Other examples are directed to pelvic therapy devices, such as various forms of incontinence, whether urinary and/or fecal such as (but not limited to) stress urinary incontinence (SUI).

FIG. 13B is a diagram schematically representing an example IMD 1419A comprising at least some of substantially the same features and attributes as the IMD 1411 in FIG. 13B, except with the IPG 1433 implemented as a microstimulator 1419B. In some examples, the microstimulator 1419B may be chronically implanted (e.g., intravasuclarly) in a head-and-neck region 1403 as shown in FIG. 13B in a manner similar to, but not limited to, the earlier described examples of intravascular placement of a microstimulator. In some examples, as part of the IMD 1419A, the microstimulator 1419B may be in wired or wireless communication with stimulation electrode 1412. In some examples, as part of the IMD 1419A, the microstimulator 1419B also may incorporate sensor 1435 or be in wireless or wired communication with a sensor 1435 located separately from a body of the microstimulator 1419B. When wireless communication is employed for sensing and/or stimulation, the microstimulator 1419B may be referred to as leadless IMD for purposes of sensing and/or stimulation. In some examples, the microstimulator 1419B may be in close proximity to a target nerve, such as the hypoglossal nerve 1405.

In some examples, the microstimulator 1419B and/or IMD 1419A may comprise at least some of substantially the same features and attributes as described and illustrated in: U.S. Patent Publication 2020/0254249, filed on Aug. 8, 2020, and entitled “MICROSTIMULATION SLEEP DISORDERED BREATHING (SDB) THERAPY DEVICE”; the PCT application published as WO 2021/242633 on Dec. 2, 2021, entitled SINGLE OR MULTIPLE NERVE STIMULATION TO TREAT SLEEP DISORDERED BREATHING; and/or U.S. Patent Publication 2020/0391028, filed on Sep. 2, 2020, and entitled “IMPLANT-ACCESS INCISION AND SENSING FOR SLEEP DISORDERED BREATHING (SDB) THERAPY DEVICE”, the entire teachings of each are hereby incorporated by reference herein in their entireties.

FIGS. 14A-18D illustrate different example variations of guide tools and/or IMDs. In some examples, the guide tool comprises a guide catheter that is used to traverse through the vasculature and the lead may be guided through the middle of the guide catheter. In other examples, the lead itself may be the guide tool having an elongate arm.

FIG. 14A illustrates an example of a guide tool that is a guide catheter having the distal elongate portion 26 and the elongate arm 24, and which may comprise at least some of substantially the same features and attributes as described by the guide tool 20 of FIG. 2. The common features and attributes are not re-described below. As shown, a lead 1555 having a stimulation element (not illustrated) may be guided through the middle of the guide catheter. In other examples, the lead 1555 may be advanced over the guide catheter, such that the lead 1555 has a greater diameter than the guide catheter and at least one conduit extending through a length of the lead 1555 and through which the guide catheter may extend.

FIGS. 14B-14E illustrates example leads, which may be the guide tool or may be guided using the guide tool.

FIG. 14B is a side view schematically illustrating a lead 1500 including a lead body 1502 having a proximal portion 1504 and a distal portion 1506, which supports a stimulation electrode array 1509. The electrode array 1509 includes electrodes 1510 spaced apart along a length of the distal portion 1506 of the lead body 1502. In some examples, lead 1500 includes an anchor 1508 at the proximal portion 1504 of lead body 1502, which is configured to maintain the position of the lead body 1502 relative to a length of the vessel through which the lead body 1502 extends. The anchor 1508 may maintain the position of the electrode array 1509 at a target location within the vessel adjacent to the target tissue.

Once implanted, an IMD for treating SBD may remain stable and endure the normal activities of the patient. The neck of a patient moves through a wide range of motion through many different positions. To counteract the potential for a lead to move back and forth within a vessel, examples include an anchoring mechanism (e.g., anchor 1508) to anchor a distal portion of a stimulation lead within a vessel at the target location relative to a target tissue. The anchoring mechanism insures that placement of the stimulation lead is maintained despite the dynamic motion and varying positions of the neck, which may otherwise cause inadvertent repositioning of the lead (relative to the target tissue) if the distal anchoring mechanisms is not present. Several examples of a distal anchoring mechanism are described and illustrated in association with FIGS. 14C-17.

FIG. 14C is a side view schematically illustrating a lead 1530 including a distal anchoring mechanism. In some examples, a lead 1530 includes a lead body 1532 having a proximal portion 1534 and a distal portion 1536, which supports a stimulation electrode array 1539. The electrode array 1539 includes surface electrodes 1540 spaced apart along a length of the distal portion 1536 of the lead body 1532. The distal portion 1536 of lead body 1532 may comprise a distal anchoring mechanism arranged as a coiled configuration 1507 and which is configured to maintain the position of the lead body 1502 relative to a length of the vein(s) through which the lead body 1532 extends. This coiled configuration acts to fix the distal portion 1536 of the lead body 1532 within the vessel at the location at which the electrodes 1540 apply an electrical stimulus. In some examples, prior to insertion of the lead 1530 into the vasculature, the distal portion 1536 is in the coiled configuration 1507. In order to install the lead 1530 into the vasculature, the distal portion 1536 is converted from the coiled configuration 1507 into a generally straight configuration by advancing a guide wire through at least the distal portion 1536 of the lead body 1532. After maneuvering the guide wire and the lead 1530 to the target location within the vasculature, the guide wire is removed proximally from the lead body 1502, which allows the distal portion 1536 to return to the coiled configuration 1507. In some examples, this “memory effect” of the coiled configuration is achieved via incorporating materials such as Nitonol or thermo-formed polyurethane into the distal portion 1536, or using other materials having memory behavior, as known by those skilled in the art, are employed to form distal portion 1536, thereby enabling the operation of coiled configuration 1507.

In some examples, as described above, a plurality of stimulation parameters (e.g., electrode polarity, amplitude, frequency, pulse width, and duration) are tested at each potential stimulation site as the stimulation lead 1530 is maneuvered through the vasculature adjacent to the target tissue. By evaluating the response at each location in the target tissue region and noting the particular value or combination of stimulation parameters that yields the best response at that potential location, one may determine the optimal stimulation site for stimulation electrode array 1539. Determining a stimulation site may be applied to anyone of the different stimulation elements configurations described herein.

FIG. 14D illustrates another example lead 1550 includes an array of ring electrodes 1552, 1554, 1556 at a distal portion of the lead 1550 and which is configured to apply stimulation to target tissue. In some examples, the array of ring electrodes 1552, 1554, 1556 directs an electrical field to a target tissue (e.g., hypoglossal nerve) spaced apart from the lead 1550 within the vessel (e.g., VCH 338). The ring electrodes 1552, 1554, 1556 may be employed in any of the other examples described above.

FIG. 14E is a perspective view schematically illustrating a lead 1560 including a distal anchoring mechanism. In some examples, the lead 1560 includes a stent structure 1562 at a distal portion of the lead 1560. The stent structure 1562 may include one or more stimulation electrodes 1564, 1566, 1568 incorporated into (or added onto) the structure (e.g., struts) of the stent. In some examples, the electrodes 1564, 1566, 1568 supported by the stent structure 1562 direct an electrical field to target tissue (e.g., hypoglossal nerve) spaced apart from the lead 1560 within the vessel (e.g., VCH). The stent structure 1562 may provide a mechanism to secure the electrodes 1564, 1566, 1568 at a target placement along a length of the vessel (through which the lead 1560 extends) corresponding to a target stimulation site of the target tissue.

In some examples, the stent structure 1562 is arranged in a collapsed state (having a diameter generally represented by A in FIG. 14E) during insertion into the vasculature and the vessel adjacent the target tissue. Once the stent structure 1562 is located at a potential stimulation site, the physician initiates conversion of the stent structure 1562 from the collapsed state to an expanded state (having a diameter generally represented by B in FIG. 14E) to contact the walls of the vessel, which anchors the electrodes in place. In some examples, during evaluation of different stimulation sites, the stent structure 1562 is temporarily expanded to test the effectiveness of a stimulation signal at a potential stimulation site and then re-collapsed to reposition the lead 1560 to place the electrodes at a different potential stimulation site. This process is repeated until an optimal stimulation site is determined, where the stent structure 1562 is re-expanded to secure and maintain the distal portion of the lead 1560 at the optimal stimulation site. The selective expansion, collapse, and final fixation of the stent structure 1562 may be performed using known techniques, such as manipulating the stent structure 1562 via rotation, pushing, and/or pulling of a guide wire.

In some examples, instead of using the coiled configuration of FIG. 14C or the stent structure of FIG. 14E, fixation of a distal portion of a stimulation lead within a vessel may be achieved via other mechanisms.

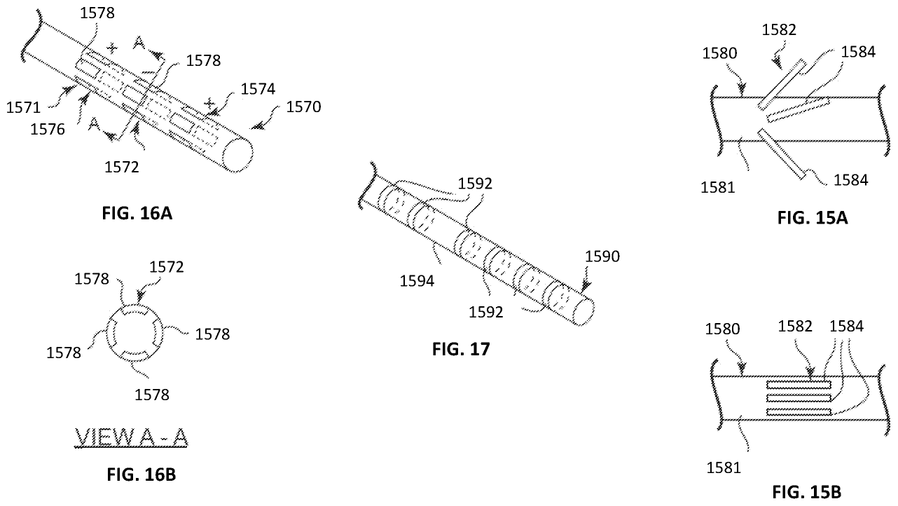

FIGS. 15A-15B schematically illustrate a stimulation lead 1580 including a distal fixation mechanism. As illustrated in FIG. 15A, a distal portion 1581 of the stimulation lead 1580 includes a distal fixation mechanism provided via an array 1582 of deployable tines 1584. In FIG. 15A, the tines 1584 are shown in a deployed configuration in which tines 1584 extend radially from the body of distal portion 1581 of lead 1580. In this position, the tines 1584 releasably engage the walls of a vessel to anchor the distal portion 1581 within the vessel. The tines 1584 may be configured to not negatively impact the integrity of the walls of the vessel.

In some examples, as the distal portion of the lead 1580 is advanced through the vasculature, a guidewire is used to position these tines 1584 into a storage position generally against an outer wall of the lead, as illustrated in FIG. 15B. After a suitable stimulation site has been determined, the tines 1584 are deployed (e.g., selectively expanded radially outward away from the outer wall of the lead as shown in FIG. 15A) to engage the walls of the vessel to anchor the distal portion 1581 of the lead 1580 at the stimulation site along that vessel (and adjacent to the target location along the target tissue). In some examples, the deployable tines 1584 are made of a polyurethane material and/or a Nitonol spring.

The array 1582 of tines 1584 may be located on distal portion 1581 of lead 1580 at a position sufficiently close to an stimulation element of lead 1580 (such as in an electrode configuration illustrated herein) to insure that the stimulation element is generally fixed within a vessel at a location corresponding to a stimulation site of target tissue.

FIGS. 16A-16B illustrates another example lead 1570. FIG. 16A is a view illustrating a stimulation lead 1570 configured to apply an electrical stimulus to target tissues. In some examples, the lead 1570 includes a series 1571 of programmable arrays 1572, 1574, 1576 of electrode portions 1578 with the respective arrays 1572, 1574, 1576 spaced apart from each other along a length of the distal portion of the lead 1570. In some examples, the electrode portions 1578 of each array extend circumferentially about an outer surface of lead 1570 in a spaced apart relationship to form a general ring-shaped configuration. In some examples, the programmable arrays 1572, 1574, 1576 direct an electrical field from a location within the vessel (e.g., ranine vein (VCH) 338) to a target tissue (e.g., hypoglossal nerve) spaced apart from the lead 1570. The arrays 1572, 1574, 1576 of electrodes may be optionally employed in other example leads described herein.

In some examples, each array 1572, 1574, 1576 of electrodes comprises two, three, four or more independent electrode portions 1578. The electrode portions 1578 may be independently programmed to stimulate the target tissue. At any given time, a stimulation signal is applied from zero, one, two, or more electrode portions 1578 of each separate array 1572, 1574, 1576. In some examples, the varied positions of the electrode portions along the length of the distal portion of the lead 1570 and circumferentially or radially about the lead 1570 enables activation of selective groups of electrode portions 1578 to produce a stimulation signal at virtually any point relative to the distal portion of lead 1570.

FIG. 17 is a view illustrating a stimulation lead 1590 configured to apply an electrical stimulus to target tissue. In some examples, a lead 1590 includes a programmable array of ring electrodes 1592 mounted at a distal portion 1594 of the lead 1590. The programmable array of ring electrodes 1592 may direct an electrical field from the location of the respective ring electrodes 1592 within the vessel (e.g., VCH 338 in FIG. 11A) to target tissue (e.g., hypoglossal nerve) spaced apart from the lead 1590. The ring electrodes 1592 may be optionally employed in any of the examples described herein. In some examples, each of the ring electrodes 1592 may be independently programmed to stimulate the target tissue. In some examples, the many varied positions of the ring electrodes 1592 along the length of the distal portion of the lead 1590 enables activation one, two, or more ring electrodes 1592 to produce a stimulation signal at virtually any point along a length of the distal portion of lead 1590. Once lead 1590 is located in the region of interest, the lead 1590 need not be maneuvered extensively distally or proximally within the vessel to position an electrode adjacent to a target stimulation site because any one or combination of the ring electrodes 1592 along the length of the distal portion of the lead are available for activation to apply a stimulation signal to the target tissue. The array of electrodes 1592 may have a length that substantially matches a majority of a length of the hypoglossal nerve, as it extends from a position near the jugular vein toward the genioglossus muscle. In some examples, the length of the array enables determining which electrodes 1592 of the array produce the most efficacious respiratory airway patency without having to reposition the array within the vasculature. In some examples, an efficacious respiratory airway patency is determined upon identifying which ring electrode 1592 or combination of ring electrodes 1592 produces a longest duration of increased airway patency, a largest size of increased airway patency, and/or a substantial reduction in apneas.

FIGS. 18A-18D illustrate example guide tools including a sensor. The guide tools 1531, 1537, 1541, 1547 in FIGS. 18A-18D may include at least some of substantially the same features and attributes as, or an example implementation of, the guide tool 20 of FIG. 2 and related examples throughout the present disclosure. The common features and attributes are not re-described below.

As shown in FIG. 18A, in some examples, the guide tool 1531 includes a sensor 1535 disposed on the elongate arm 24. The sensor 1535 may include an angle sensor, such as an impedance or conductance sensor, or a pressure sensor. The angle or pressure sensor may detect changes in impedance or pressure, which may change depending on a type of tissue surface or lack of tissue that the sensor 1535, or sensor element coupled to the sensor, is against. In some examples, the sensor 1535 may include a light sensor and/or a sound (e.g., sonographic, ultrasonic, among others) sensor.

In some examples, the sensor 1535 may not be disposed on the elongate arm 24. For example, as illustrated in FIG. 18B, the guide tool 1537 may include conductive filaments 1538 extending from or on the elongate arm 24. The conductive filaments 1538 may electrically couple to the sensor 1535 via an electrical trace 1529. In other examples, as shown in FIG. 18C, the guide tool 1541 may not include an elongate arm, and may include conductive filaments 1543 coupled to the distal elongate portion 26 of the guide tool 1541. The conductive filaments 1543 may electrically couple to the sensor 1535 via an electrical trace 1529. In other examples, as shown in FIG. 18D, the guide tool 1547 may include transducer material 1549 disposed on the elongate arm 24, and which electrically couple to the sensor 1535 via an electrical trace 1529. The conductive filaments 1538, 1543 or transducer material 1549 may electrically couple to the sensor 1535 to detect changes in impedance due to surfaces touched by the conductive filaments 1538, 1543 or transducer material 1549. The conductive filaments 1538, 1543 may be whisker-like and rotatable. In some examples, the conductive filaments 1538, 1543 may be formed of a conductive material such as metals (e.g., copper, silver, aluminum, gold, steel, brass), semiconductor material, plasmas, graphite, and conductive polymers, among other material. In some examples, the transducer material 1549 may include a piezoelectric material, such as zinc oxide, lead zirconate titanate, quartz, polyvinylidene fluoride, and crystals, among other material.

Examples are not limited to detecting changes in impedance, and the sensor 1535 may sense changes in pressure, tension, and/or other feedback at the elongate arm 24. As previously described, the change in impedance, pressure, tension, and/or other feedback (e.g., light, sound) may be used to identify an entry region, valve, and/or other topographic structure. As an example, in response to identifying an entry region, a threshold amount of force sufficient to cross or push through valve(s) may be applied to the lead, such that the elongate arm 24 may advance through the valve(s).

As implicated by the above, the IMD may include a controller, control unit, or control portion that prompts performance of designated actions. FIG. 19A is a block diagram schematically representing an example control portion 1600. In some examples, the control portion 1600 includes a controller 1602 and a memory 1604. In some examples, the control portion 1600 provides one example implementation of a control portion forming a part of, implementing, and/or managing any one of guide tools, devices, systems, assemblies, circuitry, managers, engines, functions, parameters, sensors, electrodes, and/or methods, as represented throughout the present disclosure in association with FIGS. 1-18D.

In general terms, the controller 1602 of the control portion 1600 comprises an electronics assembly 1606 (e.g., at least one processor, microprocessor, integrated circuits and logic, etc.) and associated memories or storage devices. The controller 1602 is electrically couplable to, and in communication with, the memory 1604 to generate control signals to direct operation of at least some the guide tools, devices, systems, assemblies, circuitry, managers, modules, engines, functions, parameters, sensors, electrodes, and/or methods, as represented throughout the disclosure. In some examples, these generated control signals include, but are not limited to, employing the care engine 1610 of an IMD which may be a software program stored on the memory 1604 (which may be stored on another storage device and loaded onto the memory 1604), and executed by the electronics assembly 1606 to stimulate target tissue using the stimulation element. In addition, and in some examples, these generated control signals include, but are not limited to, employing the care engine 1610 stored in the memory 1604 to at least manage care provided to the patient, for example cardiac therapy, pelvic therapy, or therapy for SDB, in at least some examples. The control portion 1600 may also be employed to operate general functions of the various care devices/systems described throughout.

In response to or based upon commands received via a user interface (e.g., user interface 1640 in FIG. 20) and/or via machine readable instructions, controller 1602 generates control signals in accordance with at least some of the examples. In some examples, controller 1602 is embodied in a general purpose computing device while in some examples, controller 1602 is incorporated into or associated with at least some of the guide tools, sensors, stimulation elements, power/control elements (e.g., IPG), devices, user interfaces, instructions, information, engines, functions, actions, and/or method, etc. as described throughout.

Regarding the controller 1602, the term “processor” shall mean a presently developed or future developed processor (or processing resources) that executes machine readable instructions contained in a memory. In some examples, execution of the machine readable instructions, such as those provided via memory 1604 of control portion 1600 cause the processor to perform the above-identified actions, such as operating controller 1602 to implement the sensing, monitoring, identifying the care cycle, stimulation, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1604. The machine readable instructions may comprise a sequence of instructions, a machine learning model, or the like. In some examples, memory 1604 comprises a machine readable tangible medium providing non-volatile storage of the machine readable instructions executable by a process of controller 1602. The machine readable tangible medium may sometimes be referred to as, and/or comprise at least a portion of, a computer program product. Hard wired circuitry may be used in place of or in combination with machine readable instructions to implement the functions described. For example, controller 1602 may be embodied as part of at least one application-specific integrated circuit (ASIC), at least one field-programmable gate array (FPGA), and/or the like. In some examples, the controller 1602 is not limited to any specific combination of hardware circuitry and machine readable instructions, nor limited to any particular source for the machine readable instructions executed by the controller 1602.

In some examples, control portion 1600 may be entirely implemented within or by a stand-alone device.

In some examples, the control portion 1600 may be partially implemented in one of the guide tools, sensors, stimulation elements, IMDs (or portions thereof), etc. and partially implemented in a computing resource (e.g., at least one external resource) separate from, and independent of, the IMDs (or portions thereof) but in communication with the IMDs (or portions thereof). In some examples, control portion 1600 may be implemented via a server accessible via the cloud and/or other network pathways. In some examples, the control portion 1600 may be distributed or apportioned among multiple devices or resources such as among a server, an apnea treatment device (or portion thereof), and/or a user interface.

In some examples, control portion 1600 includes, and/or is in communication with, a user interface 1640 as shown in FIG. 20.

FIG. 19B is a diagram schematically illustrating example arrangements of a control portion 1620 by which the control portion 1600 (FIG. 19A) may be implemented. In some examples, control portion 1620 is implemented within or by an IPG 1625, which has at least some of substantially the same features and attributes as the IPG (e.g., power/control element) as previously described. In some examples, control portion 1620 is implemented within or by a remote control 1630 (e.g., a programmer) external to the patient's body, such as a patient control 1632 and/or a physician control 1634. In some examples, the control portion 1600 is partially implemented in the IPG 1625 and partially implemented in the remote control 1630 (at least one of patient control 1632 and physician control 1634).

FIG. 20 is a block diagram schematically representing a user interface 1640. In some examples, user interface 1640 forms part of and/or is accessible via a device external to the patient and by which the IMD may be at least partially controlled and/or monitored. The external device which hosts the user interface 1640 may be a patient remote (e.g., 1632 in FIG. 19B), a physician remote or physical controller associated with and/or for operating a guide tool (e.g., 1634 in FIG. 19B), imaging equipment, and/or a clinician portal. In some examples, user interface 1640 comprises a user interface or other display that provides for the simultaneous display, activation, and/or operation of at least some of the guide tools, sensors, stimulation elements, power/control elements (e.g., pulse generators), devices, user interfaces, instructions, information, modules, engines, functions, actions, and/or method, etc., as described in association with FIGS. 1-18B. In some examples, at least some portions or aspects of the user interface 1640 are provided via a graphical user interface (GUI), and may comprise a display 1644 and input 1642.

FIG. 21 is a block diagram 1650 schematically representing example implementations by which an IMD 1660 may communicate wirelessly with external circuitry outside the patient. As described above, the controller and/or control portion of the IMD 1660 may be implemented by components of the IMD 1660, components of external circuitry, such as external devices (e.g., mobile device 1670, patient remote control 1674, a clinician programmer 1676, and a patient management tool 1680), and combinations thereof. As shown in FIG. 20, the IMD 1660 may communicate with at least one of patient application 1672 on a mobile device 1670, a patient remote control 1674, a clinician programmer 1676, and a patient management tool 1680. The patient management tool 1680 may be implemented via a cloud-based portal 1683, the patient application 1672, and/or the patient remote control 1674. Among other types of data, these communication arrangements enable the IMD 1660 to communicate, display, manage, etc. the data events, as well as to allow for adjustment to the various elements, portions, etc. of the example devices and methods if and where desired.

The various ranges provided herein include the stated range and any value or sub-range within the stated range. Furthermore, when “about” is utilized to describe a value, this includes, refers to, and/or encompasses variations (up to +/−10%) from the stated value.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

Example A1. A method, comprising: establishing, via a guide tool, a stimulation element in a first position within a first vessel of a vasculature which is in communication with a second vessel of the vasculature; identifying, via angularly positioning of an elongate arm relative to a distal elongate portion of the guide tool, an entry region into the second vessel from the first vessel; and maneuvering the stimulation element through the entry region and to a second position within the second vessel via the guide tool, the second position being adjacent to a target tissue.

Example A2. The method of example A1, further comprising transvascularly delivering stimulation to the target tissue via the stimulation element.

Example A3. The method of example A1, further comprising angularly positioning the elongate arm relative to the distal elongate portion of the guide tool to at least one of: a tilt angle relative to the distal elongate portion of the guide tool; and/or a rotational angle relative to a longitudinal axis of the distal elongate portion.

Example A4. The method of example A1, wherein maneuvering the stimulation element comprises inserting the guide tool along a pathway through the vasculature from the first vessel to the second vessel, wherein the entry region includes a junction of the first vessel and the second vessel through which the stimulation element is inserted and advanced.

Example A5. The method of example A4, wherein the second vessel extends at a first angle relative to the first vessel, the first angle comprising: an acute angle, a perpendicular angle, and/or an obtuse angle.

Example A6. The method of example A1, wherein identifying the entry region into the second vessel and maneuvering the stimulation element through the entry region and to the second position within the second vessel comprises advancing the distal elongation portion of the guide tool to the second position along a pathway through the vasculature by identifying surfaces within the vasculature via the angularly positioning of the elongate arm.

Example A7. The method of example A6, wherein the surfaces are associated with topographic structures including the entry region and valves within the second vessel, the surfaces being identified by: identifying the surfaces within the vasculature via the angularly positioning of the elongate arm and palpable contact of the elongate arm with the surfaces while the elongate arm is at a tilt angle relative to the distal elongate portion of the guide tool; and in response, maneuvering the guide tool along the pathway through the vasculature and to the second position within the second vessel.

Example A8. The method of example A7, wherein the palpable contact of the elongate arm with the surfaces is transmitted from the elongate arm to a handle of the guide tool.

Example A9. The method of example A6, wherein identifying the surfaces includes using sensor signals from a sensor or sensor element associated with the elongate arm, wherein the sensor or sensor element is selected from the group consisting of: an angle sensor, a pressure sensor, a light sensor, a sound sensor, conductive filaments, transducer material, and combinations thereof.

Example A10. The method of example A1, wherein maneuvering the stimulation element through the entry region and to the second position within the second vessel comprises: advancing the distal elongate portion of the guide tool proximate to a target location within the second vessel adjacent to the target tissue via a pathway through the second vessel; and positioning the stimulation element proximate to the target location via the guide tool.

Example A11. The method of example A1, further comprising: activating the angularly positioning of the elongate arm in response to establishing the distal elongate portion of the guide tool at the first position within the first vessel by placing the elongate arm at a tilt angle relative to the distal elongate portion of the guide tool; and angularly positioning the elongate arm of the guide tool by at least one of: (i) titling to the tilt angle relative to the distal elongate portion of the guide tool in a first direction and to a second tilt angle in an opposite second direction to identify at least one surface of the vasculature; and/or (ii) rotating about a rotational angle relative to a longitudinal axis of the distal elongate portion, while the elongate arm is at the tilt angle, to identify at least one surface of the vasculature.

Example A12. The method of example A11, wherein activating the angularly positioning of the elongate arm comprises: at least one of mechanically, electrically, and/or magnetically causing the elongate arm to transition from a first state to a second state associated with an angular position of the elongate arm; and in the second state, to move to the tilt angle relative to the distal elongate portion.

Example A13. The method of example A12, further comprising transitioning back to the first state in response to sensed pressure indicative of the elongate arm contacting a valve within the second vessel.

Example A14. The method of example A1, wherein angularly positioning the elongate arm comprises at least one of: positioning the elongate arm 180 degrees relative to a longitudinal axis of the distal elongate portion of the guide tool by moving the elongate arm to a tilt angle with respect to the distal elongate portion in a first direction and to the tilt angle with respect to the distal elongate portion in an opposite second direction; and/or rotating the elongate arm to at least one degree position within a 360 degree range about the longitudinal axis of the distal elongate portion, while the elongate arm is at the tilt angle with respect to the distal elongate portion.

Example A15. The method of example A1, wherein maneuvering the stimulation element through the entry region and to the second position within the second vessel comprising advancing the elongate arm through the entry region and through at least one valve within the second vessel.

Example A16. The method of example A1, wherein the distal elongate portion of the guide tool further comprises a junction between the elongate arm and the distal elongate portion, the method further comprising: controlling the angularly positioning of the elongate arm via the junction, wherein the junction is selected from: (i) a hinge joint to permit tilting of the elongate arm; (ii) a pivot joint to permit rotation the elongate arm; and (iii) a ball joint to permit tilting and rotation of the elongate arm. And, wherein the elongate arm extends from the junction of the guide tool and the elongate arm comprises at least one of: a length that is less than a length of a body of the guide tool and/or the distal elongate portion; and/or a greater rigidity than the distal elongate portion.

Example A17. The method of example A1, wherein: identifying the entry region into the second vessel comprises: tilting the elongate arm to a tilt angle with respect to the distal elongate portion of the guide tool; and while at the tilt angle, rotating the elongate arm at a rotational angle relative to a longitudinal axis of the distal elongate portion of the guide tool to identify the entry region via contact of the elongate arm with surfaces of the vasculature. And, in response to the identification, maneuvering the stimulation element through the entry region and to the second position in the second vessel comprises: advancing the distal elongate portion of the guide tool through the entry region and into the second vessel; and advancing the distal elongate portion of the guide tool through at least one valve of the second vessel and to a target location within the second vessel adjacent to the target tissue.

Example A18. The method of example A17, wherein advancing the distal elongate portion of the guide tool through the at least one valve comprises: opening the at least one valve and advancing the elongate arm through the at least one valve via pressure applied by the elongate arm to the at least one valve.

Example A19. The method of example A1, wherein maneuvering the stimulation element through the entry region and to the second position in the second vessel comprises at least one of: advancing the guide tool into and through a subclavian vein, into and through a jugular vein, into and through a vein trunk, and into a vena comitante hypoglossi; and/or advancing the guide tool into and through a common facio-lingual branch, into and through a lingual vein, and into a ventral lingual vein.

Example A20. The method of example A1, wherein: the target tissue comprises a hypoglossal nerve and the second vessel comprises a vena comitante hypoglossi; and maneuvering the stimulation element through the entry region and to the second position in the second vessel comprises: advancing the distal elongate portion of the guide tool into the entry region of the vena comitante hypoglossi and through at least one valve of the vena comitante hypoglossi.

Example A21. The method of example A1, wherein the target tissue comprises an upper airway-related tissue, the method further comprising: transvascularly delivering stimulation to the target tissue via the stimulation element to treat sleep disordered breathing.

Example A22. The method of example A1, wherein: the guide tool comprises a lead comprising a proximal portion and a distal elongate portion, with an elongate portion therebetween and including the distal elongate portion, the stimulation element being disposed on or proximate to the distal elongate portion of the lead, and the stimulation element including at least one electrode; and wherein establishing the stimulation element in the first position within the first vessel of the vasculature and maneuvering the stimulation element to the second position within the second vessel comprises: inserting the lead within the first vessel of the vasculature; and advancing the distal elongate portion of the lead including the stimulation element to the second position proximate to a target location within the second vessel adjacent to the target tissue.

Example A23. The method of example A22, further comprising inserting an implantable pulse generator (IPG) subcutaneously within a body of a patient and connecting the proximal portion of the lead to the IPG.

Example A24. The method of example A1, wherein: the guide tool comprises a guide catheter comprising a proximal portion and a distal portion, with an elongate portion therebetween and including the distal elongate portion; and establishing the stimulation element in the first position within the first vessel of the vasculature of the vasculature and maneuvering the stimulation element to the second position within the second vessel comprises: inserting the guide catheter within the first vessel of the vasculature; advancing the distal elongate portion of the guide catheter to a target location within the second vessel adjacent to the target tissue; and maneuvering a lead comprising the stimulation element through the guide catheter from the proximal portion through the distal elongate portion and to the second position proximate to the target location within the second vessel.

Example A25. The method of example A24, further comprising introducing a contrast agent within the vasculature via the guide catheter while advancing the distal elongate portion of the guide catheter proximate to the target location.

Example A26. The method of example A1, wherein the stimulation element is maneuvered to the second position without use of a contrast agent and, optionally, without use of an imaging system to detect the contrast agent.

Example A27. The method of example A1, wherein the stimulation element comprises a microstimulator comprising at least one electrode.

Example A28. The method of example A27, wherein maneuvering the stimulation element through the entry region and to the second position comprises at least one of: removably attaching the microstimulator to the guide tool; or releasing the microstimulator from the guide tool in response to the microstimulator being in the second position within the second vessel proximate to the target tissue.

Example A29. The method of example A28, further comprising: maintaining the microstimulator in close proximity to the target tissue by anchoring the microstimulator relative to the second vessel via at least one of: arranging the microstimulator as at least part of a coil structure movable between a generally straight state during advancement through the vasculature and an expanded, coiled state for stimulating the target tissue; and/or arranging the microstimulator as at least part of a stent structure movable between a generally collapsed state during advancement through the vasculature and an expanded state at the second position for stimulating the target tissue.

Example A30. The method of example A1, wherein identifying the entry region into the second vessel and maneuvering the stimulation element to the second position within the second vessel comprises: placing the guide tool in a first state in which the elongate arm is axially aligned with the distal elongate portion of the guide tool such that the elongate arm and the distal elongate portion are positioned generally straight with respect to one another; and activating the elongate arm and placing the guide tool in a second state in which the elongate arm is at a tilt angle with respect to the distal elongate portion and is axially unaligned with the distal elongate portion such that the elongate arm and the distal elongate portion are positioned at the tilt angle with respect to one another.

Example A31. The method of example A1, wherein the stimulation element comprises a first stimulation element, and the guide tool comprises a first guide tool, the method further comprising: establishing, via a second guide tool, a second stimulation element in a third position within the first vessel or a fourth vessel of the vasculature which is in communication with a third vessel of the vasculature; identifying, via angular positioning of an elongate arm of a distal elongate portion of the second guide tool, an entry region into the third vessel from the first vessel or the fourth vessel; and maneuvering the stimulation element through the entry region and to a fourth position within the third vessel via the second guide tool, the fourth position being proximate to the target tissue.

Example A32. The method of example A1, further comprising positioning the stimulation element within the second vessel at the second position in close proximity to a hypoglossal nerve and within a neck region of a patient.

Example A33. The method of example A1, further comprising: implanting an implantable pulse generator (IPG) subcutaneously at a pectoral, non-vasculature location within a body of a patient; connecting a lead to the IPG, wherein the lead includes the stimulation element that is in electrical communication with the IPG; and wherein identifying the entry region into the second vessel and maneuvering the stimulation element through the entry region and to the second position within the second vessel comprises: inserting the lead into the first vessel that connects to the second vessel; and advancing the lead into at least the entry region of the second vessel and through a plurality of valves of the second vessel via the elongate arm, wherein the guide tool comprises the lead.

Example A34. The method of example A1, wherein the stimulation element is maneuvered to the second position via the guide tool using a single entry region to the vasculature.

Example A35. The method of example A1, further comprising: detecting a sleep disordered breathing (SDB) event for a patient; and transvascularly delivering the stimulation via the stimulation element in response to the detection of the SDB event, wherein transvascularly delivering the stimulation element to the target tissue causes an increase or maintaining of patency of an upper airway of the patient.

Example B1. A guide tool comprising: a body comprising a proximal portion, a distal portion comprising an elongate arm, and an elongate portion between the proximal portion and distal portions; the elongate portion comprising a distal elongate portion coupled to the elongate arm; the elongate arm being movable to different angular positions relative to the distal elongate portion of the guide tool.

Example B2. The guide tool of example B1, wherein the elongate arm is movable among or between the different angular positions and according a degree of freedom or multiple degrees of freedom.

Example B3. The guide tool of example B1, wherein the elongate arm being movable to the different angular positions relative to the distal elongate portion of the guide tool comprises at least one of: a tilt angle relative to the distal elongate portion of the guide tool; and/or a rotational angle relative to a longitudinal axis of the distal elongate portion.

Example B4. The guide tool of example B1, wherein at least one of: the proximal portion comprises a handle; and/or the distal elongate portion comprises a linear segment of the elongate portion of the body of the guide tool that is proximate and/or coupled to the elongate arm, and that remains aligned with the elongate arm when the elongate arm is at least relatively straight or non-angled with respect to a longitudinal axis of the distal elongate portion.

Example B5. The guide tool of example B1, wherein the guide tool is shaped such that the elongate portion and the elongate arm are pushable and torquable, and the elongate arm is steerable, and the guide tool is maneuverable within and/or through the vasculature.

Example B6. The guide tool of example B5, wherein the guide tool is insertable along a pathway through a vasculature from a first vessel to a second vessel, wherein the entry region includes a junction of the first vessel and the second vessel through which the stimulation element is inserted and advanced.

Example B7. The guide tool of example B6, wherein the second vessel extends at a first angle relative to the first vessel, the first angle comprising: an acute angle, a perpendicular angle, or an obtuse angle.

Example B8. The guide tool of example B1, wherein the guide tool is to: establish a stimulation element in a first position within a first vessel of a vasculature which is in communication with a second vessel of the vasculature; identify, via the angular position of the elongate arm relative to the distal elongate portion of the guide tool, an entry region into the second vessel from the first vessel; and maneuver the stimulation element through the entry region and to a second position within the second vessel via the guide tool, the second position being adjacent to a target tissue.

Example B9. The guide tool of example B8, wherein the elongate arm is to identify the entry region into the second vessel and maneuver the stimulation element through the entry region and to the second position within the second vessel comprises advancing the distal elongation portion of the guide tool to the second position along a pathway through the vasculature by identifying surfaces within the vasculature via the angular positioning of the elongate arm.

Example B10. The guide tool of example B9, wherein the surfaces are associated with topographic structures including the entry region and valves within the second vessel, the surfaces being identified via the angular position of the elongate arm and palpable contact of the elongate arm with the surfaces while the elongate arm is at a tilt angle relative to the distal elongate portion of the guide tool; and the guide tool is maneuverable along the pathway through the vasculature and to the second position within the second vessel.

Example B11. The guide tool of example B10, wherein the elongate arm is to transmit the palpable contact with surfaces to a handle of the guide tool.

Example B12. The guide tool of example B9, further comprising a sensor or sensor element associated with the elongate arm which provides sensor signals indicative of the surfaces, wherein the sensor or sensor element is selected from the group consisting of: an angle sensor, a pressure sensor, a light sensor, a sound sensor, conductive filaments, transducer material, and combinations thereof.

Example B13. The guide tool of example B8, wherein in the guide tool is to maneuver the stimulation element through the entry region and to the second position within the second vessel comprises: the distal elongate portion of the guide tool being advanced to proximate to a target location within the second vessel adjacent to the target tissue via a pathway through the second vessel; and the stimulation element being positioned proximate to the target location via the guide tool.

Example B14. The guide tool of example B8, wherein the different angular positions of the elongate arm are activatable, wherein in response to establishing the distal elongate portion of the guide tool at the first position within the first vessel by placing the elongate arm at a tilt angle relative to the distal elongate portion of the guide tool; and angularly positioning the elongate arm of the guide tool by at least one of: titling to the tilt angle relative to the distal elongate portion of the guide tool in a first direction and to a second tilt angle in an opposite second direction to identify at least one surface of the vasculature; and/or rotating about a rotational angle relative to a longitudinal axis of the distal elongate portion, while the elongate arm is at the tilt angle, to identify at least one surface of the vasculature.

Example B15. The guide tool of example B1, wherein the guide tool is configured to be in different states associated with the different angular positions of the elongate arm with respect to the distal elongate portion.

Example B16. The guide tool of example B1, wherein the proximal portion further comprises a mechanism to activate the angular positioning of the elongate arm, the mechanism being at least one of mechanical, electrical, and magnetic and causing the elongate arm to transition from a first state to a second state associated with an respective angular position of the elongate arm; and/or in the second state, to move to the tilt angle relative to the distal elongate portion.

Example B17. The guide tool of example B16, wherein the elongate arm is to transition back to the first state in response to sensed pressure indicative of the elongate arm contacting a valve within the second vessel.

Example B18. The guide tool of example B1, wherein the guide arm being movable to the different angular position comprises at least one of: the elongate arm being positioned 180 degrees relative to a longitudinal axis of the distal elongate portion of the guide tool via movement of the elongate arm to a tilt angle with respect to the elongate portion in a first direction and to the tilt angle with respect to the elongate portion in an opposite second direction; and/or the elongate arm being rotated to at least one degree position within a 360 degree range about the longitudinal axis of the distal elongate portion, while the elongate arm is at the tilt angle with respect to the distal elongate portion.

Example B19. The guide tool of example B8, wherein the guide tool is to maneuver the stimulation element through the entry region and to the second position within the second vessel via advancing the elongate arm through the entry region and through at least one valve within the second vessel.

Example B20. The guide tool of example B1, wherein the distal elongate portion further comprises a junction between the elongate arm and the distal elongate portion, the junction to control the different angular positions of the elongate arm, wherein the junction is selected from: a hinge joint to permit tilting of the elongate arm; a pivot joint to permit rotation the elongate arm; and a ball joint to permit tilting and rotation of the elongate arm; and wherein the elongate arm extends from the junction of the guide tool and the elongate arm comprises at least one of: a length that is less than a length of a body of the guide tool and/or the distal elongate portion; and/or a greater rigidity than the distal elongate portion.

Example B21. The guide tool of example B20, wherein the junction releasably retains the different angular positions of the elongate arm.

Example B22. The guide tool of example B8, wherein the guide tool is to: tilt the elongate arm to a tilt angle with respect to the distal elongate portion of the guide tool to identify the entry region into the second vessel; while at the tilt angle, rotate the elongate arm at a rotational angle relative to a longitudinal axis of the distal elongate portion of the guide tool to identify the entry region via contact of the elongate arm with surfaces of the vasculature; and in response to the identification, maneuver the stimulation element through the entry region and to the second position in the second vessel comprising: the distal elongate portion of the guide tool being advanced through the entry region and into the second vessel; and the distal elongate portion of the guide tool being advanced through at least one valve of the second vessel and to a target location within the second vessel adjacent to the target tissue.

Example B23. The guide tool of example B22, wherein the distal elongate portion of the guide tool being advanced through the at least one valve comprises opening the at least one valve and advancing the elongate arm through the at least one valve via pressure applied by the elongate arm to the at least one valve.

Example B24. The guide tool of example B22, wherein the guide tool to maneuver the stimulation element through the entry region and to the second position in the second vessel comprises at least one of: the guide tool being advanced into and through a subclavian vein, into and through a jugular vein, into and through a vein trunk, and into a vena comitante hypoglossi; and/or the guide tool being advanced into and through a common facio-lingual branch, into and through a lingual vein, and into a ventral lingual vein.

Example B25. The guide tool of example B1, wherein: the target tissue comprises a hypoglossal nerve and the second vessel comprises a vena comitante hypoglossi; and wherein the guide tool to maneuver the stimulation element through the entry region and to the second position in the second vessel comprises: the distal elongate portion of the guide tool being advanced into the entry region of the vena comitante hypoglossi and through at least one valve of the vena comitante hypoglossi.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method, comprising:
establishing, via a guide tool, a stimulation element in a first position within a first vessel of a vasculature which is in communication with a second vessel of the vasculature;
identifying, via angularly positioning of an elongate arm relative to a distal elongate portion of the guide tool, an entry region into the second vessel from the first vessel; and
maneuvering the stimulation element through the entry region and to a second position within the second vessel via the guide tool, the second position being adjacent to a target tissue.

2. The method of claim 1, further comprising transvascularly delivering stimulation to the target tissue via the stimulation element.

3. The method of claim 1, further comprising angularly positioning the elongate arm relative to the distal elongate portion of the guide tool to at least one of:
a tilt angle relative to the distal elongate portion of the guide tool; or
a rotational angle relative to a longitudinal axis of the distal elongate portion.

4. The method of claim 1, wherein maneuvering the stimulation element comprises inserting the guide tool along a pathway through the vasculature from the first vessel to the second vessel, wherein the entry region includes a junction of the first vessel and the second vessel through which the stimulation element is inserted and advanced.

5. The method of claim 4, wherein the second vessel extends at a first angle relative to the first vessel, the first angle comprising:
an acute angle, a perpendicular angle, or an obtuse angle.

6. The method of claim 1, wherein identifying the entry region into the second vessel and maneuvering the stimulation element through the entry region and to the second position within the second vessel comprises advancing the distal elongation portion of the guide tool to the second position along a pathway through the vasculature by identifying surfaces within the vasculature via the angularly positioning of the elongate arm.

7. The method of claim 6, wherein the surfaces are associated with topographic structures including the entry region and valves within the second vessel, the surfaces being identified by:
identifying the surfaces within the vasculature via the angularly positioning of the elongate arm and palpable contact of the elongate arm with the surfaces while the elongate arm is at a tilt angle relative to the distal elongate portion of the guide tool; and
in response, maneuvering the guide tool along the pathway through the vasculature and to the second position within the second vessel.

8. The method of claim 7, wherein the palpable contact of the elongate arm with the surfaces is transmitted from the elongate arm to a handle of the guide tool.

9. The method of claim 6, wherein identifying the surfaces includes using sensor signals from a sensor or sensor element associated with the elongate arm, wherein the sensor or sensor element is selected from the group consisting of:
an angle sensor, a pressure sensor, a light sensor, a sound sensor, conductive filaments, transducer material, and combinations thereof.

10. The method of claim 1, wherein maneuvering the stimulation element through the entry region and to the second position within the second vessel comprises:
advancing the distal elongate portion of the guide tool proximate to a target location within the second vessel adjacent to the target tissue via a pathway through the second vessel; and
positioning the stimulation element proximate to the target location via the guide tool.

11. The method of claim 1, further comprising:
activating the angularly positioning of the elongate arm in response to establishing the distal elongate portion of the guide tool at the first position within the first vessel by placing the elongate arm at a tilt angle relative to the distal elongate portion of the guide tool; and
angularly positioning the elongate arm of the guide tool by at least one of:
titling to the tilt angle relative to the distal elongate portion of the guide tool in a first direction and to a second tilt angle in an opposite second direction to identify at least one surface of the vasculature; or
rotating about a rotational angle relative to a longitudinal axis of the distal elongate portion, while the elongate arm is at the tilt angle, to identify at least one surface of the vasculature.

12. The method of claim 11, wherein activating the angularly positioning of the elongate arm comprises:
at least one of mechanically, electrically, and magnetically causing the elongate arm to transition from a first state to a second state associated with an angular position of the elongate arm; and
in the second state, to move to the tilt angle relative to the distal elongate portion.

13. The method of claim 12, further comprising transitioning back to the first state in response to sensed pressure indicative of the elongate arm contacting a valve within the second vessel.

14. The method of claim 1, wherein angularly positioning the elongate arm comprises at least one of:
positioning the elongate arm 180 degrees relative to a longitudinal axis of the distal elongate portion of the guide tool by moving the elongate arm to a tilt angle with respect to the distal elongate portion in a first direction and to the tilt angle with respect to the distal elongate portion in an opposite second direction; or
rotating the elongate arm to at least one degree position within a 360 degree range about the longitudinal axis of the distal elongate portion, while the elongate arm is at the tilt angle with respect to the distal elongate portion.

15. The method of claim 1, wherein maneuvering the stimulation element through the entry region and to the second position within the second vessel comprising advancing the elongate arm through the entry region and through at least one valve within the second vessel.

16. The method of claim 1, wherein the distal elongate portion of the guide tool further comprises a junction between the elongate arm and the distal elongate portion, the method further comprising:
controlling the angularly positioning of the elongate arm via the junction, wherein the junction is selected from:
a hinge joint to permit tilting of the elongate arm;
a pivot joint to permit rotation the elongate arm; and
a ball joint to permit tilting and rotation of the elongate arm; and
wherein the elongate arm extends from the junction of the guide tool and the elongate arm comprises at least one of:
a length that is less than a length of a body of the guide tool and/or the distal elongate portion; or
a greater rigidity than the distal elongate portion.

17. The method of claim 1, wherein:
identifying the entry region into the second vessel comprises:
tilting the elongate arm to a tilt angle with respect to the distal elongate portion of the guide tool; and
while at the tilt angle, rotating the elongate arm at a rotational angle relative to a longitudinal axis of the distal elongate portion of the guide tool to identify the entry region via contact of the elongate arm with surfaces of the vasculature; and
in response to the identification, maneuvering the stimulation element through the entry region and to the second position in the second vessel comprises:
advancing the distal elongate portion of the guide tool through the entry region and into the second vessel; and
advancing the distal elongate portion of the guide tool through at least one valve of the second vessel and to a target location within the second vessel adjacent to the target tissue.

18. The method of claim 17, wherein advancing the distal elongate portion of the guide tool through the at least one valve comprises:
opening the at least one valve and advancing the elongate arm through the at least one valve via pressure applied by the elongate arm to the at least one valve.

19. The method of claim 1, wherein maneuvering the stimulation element through the entry region and to the second position in the second vessel comprises at least one of:
advancing the guide tool into and through a subclavian vein, into and through a jugular vein, into and through a vein trunk, and into a vena comitante hypoglossi; or
advancing the guide tool into and through a common facio-lingual branch, into and through a lingual vein, and into a ventral lingual vein.

20. The method of claim 1, wherein:
the target tissue comprises a hypoglossal nerve and the second vessel comprises a vena comitante hypoglossi; and
maneuvering the stimulation element through the entry region and to the second position in the second vessel comprises:
advancing the distal elongate portion of the guide tool into the entry region of the vena comitante hypoglossi and through at least one valve of the vena comitante hypoglossi.
